(19) United States
(12) Patent Application Publication
(10) Pub. No.: US 2018/0022808 A1
(43) Pub. Date: Jan. 25, 2018

(54) ANTIBODY CONSTRUCT

(71) Applicant: MEDIZINISCHE UNIVERSITÄT WIEN, Vienna (AT)

(72) Inventors: Christoph Madritsch, Vienna (AT); Sabine Flicker, Vienna (AT); Rudolf Valenta, Theresienfeld (AT); Elisabeth Gader-Maier, Vienna (AT); Julia Eckl-Dorna, Vienna (AT); Verena Niederberger, Vienna (AT); Katharina Blatt, Groß-Enzersdorf (AT); Peter Valent, Vienna (AT); Isabella Ellinger, Vienna (AT)

(21) Appl. No.: 15/550,073

(22) PCT Filed: Feb. 10, 2016
(86) PCT No.: PCT/EP2016/052859

§ 371 Date: Aug. 10, 2017

(30) Foreign Application Priority Data
Feb. 10, 2015 (EP) 15154521.7

(51) Int. Cl. C07K 16/28; C07K 16/16; C07K 16/46; A61K 39/395

(52) U.S. Cl. CPC C07K 16/2821; A61K 39/395; C07K 16/16; C07K 16/46; C07K 2317/31; C07K 2317/55; C07K 2317/70

(57) ABSTRACT

The present invention relates to an antibody construct comprising at least one antigen binding site specific for ICAM1 and at least one antigen binding site specific for at least one allergen.

The present invention relates to an antibody construct comprising an antigen binding site specific for an allergen and uses thereof in the treatment and prevention of allergies.

IgE-associated allergies affect more than 25% of the population and hence represent a global health problem. Immediate allergic symptoms are caused by cross-linking of FcεRI on mast cells and basophils by IgE-allergen immune complexes. Current treatments to prevent or to reduce allergic symptoms, especially allergen-specific immunotherapy (SIT), aim to impede such a crosslinking and/or the binding of IgE molecules to allergens by induction of allergen-specific IgG antibodies. These treatments usually involve the administration of allergens, hypoallergenic variants of allergens or fragments of these polypeptides and proteins to patients. Since these molecules are usually administered by parenteral and thus invasive routes it is an object of the present invention to provide alternative means for the treatment or prevention of allergies or for the alleviation of allergic symptoms.

Therefore, the present invention relates to an antibody construct comprising at least one antigen binding site specific for ICAM1 and at least one antigen binding site specific for at least one allergen.

In order to reach immune cells which are resident in the target organs of allergic inflammation allergens must cross the epithelial barrier. Disturbances of the epithelial barrier function by endogenous factors (e.g. mutations in the filaggrin gene or chronic inflammation) and exogenous factors (e.g. damage of the epithelium by the proteolytic activity of allergens, rhinovirus infections and tobacco smoking) foster trans-epithelial allergen migration and subsequent allergic inflammation and may contribute to increases of local and systemic allergen-specific immune responses. Therefore, measures for improving barrier function, such as topical treatments of skin and nasal mucosa may reduce allergen uptake and allergic inflammation. It turned out that antibody constructs comprising binding sites specific for ICAM1 and an allergen are able to reduce significantly the allergen systemic uptake (i.e. transepithelial allergen migration) by capturing the allergen on the surface of epithelial cells, in particular respiratory, conjunctival and gastrointestinal epithelial cells. This prevents trans-epithelial allergen migration and, as a consequence, reduces allergic inflammation.

Intercellular adhesion molecule 1 (ICAM1; human ICAM1 has the sequence UniProt Acc. No. P05362), also known as CD54, is a type of intercellular adhesion molecule present in the membranes of, e.g., endothelial cells. ICAM1 present on the surface of cells is able to bind leukocytes enabling them to transmigrate into tissues. It was further characterized to be a site for the cellular entry of human rhinovirus. ICAM1 is markedly augmented by inflammatory mediators, including TNF-α, IL-1α and β, IFN-γ and endotoxins.

It has been reported that ICAM1 is highly expressed on the respiratory epithelium of allergic patients, for instance. Although ICAM1 is described to be responsible for the migration of molecules through tissues, it was surprisingly found that an antibody construct comprising an antigen binding site specific for ICAM1 and an antigen binding site specific for an allergen is useful for capturing allergens at the sites of tissue entry, thus preventing or significantly reducing the transmigration of allergens bound to said construct. The antibody construct of the present invention binds stably to the allergen as well as to ICAM1 on epithelial cells, in particular respiratory, conjunctival and gastrointestinal epithelial cells, and prevents trans-epithelial allergen migration. The complex comprising the antibody construct of the present invention and allergen formed on epithelial cells prevents the internalization of the allergen bound on the surface of these cells. Consequently, allergic inflammation can be significantly reduced.

The antibody construct of the present invention is able to trap the allergens on the respiratory epithelium, the epithelium of the conjunctiva and the epithelium of the gastrointestinal tract, for instance, thus preventing its transmigration. This is possible due to the use of at least one antigen binding site specific for ICAM1 which allows the antibody construct to remain on the (respiratory, conjunctival and/or gastrointestinal) epithelium for at least 6 hours, preferably at least 12 hours, more preferably for at least 24 hours, even more preferably for at least 36 hours. The ICAM1 binding molecules of the present invention which are part of the antibody construct of the present invention stay on the surface (respiratory, conjunctival and/or gastrointestinal) epithelium and do essentially not promote transmigration. Such ICAM1 binding molecules can be identified, for instance, using transmigration assays known to a person skilled in the art. These transmigration assays are preferably performed using respiratory, conjunctival and gastrointestinal epithelial cells.

In a particularly preferred embodiment of the present invention the epithelium, which is targeted by the antibody construct of the present invention, does not include endothelial cells and the endothelium.

The antibody construct of the present invention comprises at least one antigen binding site for ICAM1 and at least one antigen binding site for an allergen. In a preferred embodiment of the present invention the antibody construct of the present invention may comprise at least two, preferably at least three, more preferably at least five, antigen binding sites for ICAM1. In a further preferred embodiment of the present invention the antibody construct of the present invention may comprise at least two, preferably at least three, more preferably at least five, antigen binding sites for at least one, preferably at least two, more preferably at least five, allergen(s).

If the antibody construct of the present invention comprises more than one antigen binding sites for an allergen, the binding sites may be specific for one or more allergens. Thus, an antibody construct of the present invention may comprise antigen binding sites for one specific allergen or for more allergens. Such an antibody construct can be used to capture not only one allergen.

“Antibody construct” as used herein refers to any molecule, conjugate or complex comprising at least two different antigen binding sites enabling the construct to bind to, to interact with or to recognize the target molecules ICAM1 and an allergen.

“Conjugate” as used herein refers to at least a first antigen binding site or a first immunoglobulin or immunoglobulin fragment covalently bound to at least a second antigen binding site or a second immunoglobulin or immunoglobulin fragment, wherein the conjugate is able to bind to ICAM1 and an allergen. The parts of the conjugate of the present invention can be bound directly to each other or via a linker or spacer. A “linker” or “spacer” can be any chemical moiety that is capable of linking an antigen binding site, immunoglobulin or immunoglobulin fragment to at least another antigen binding site, immunoglobulin or immunoglobulin fragment in a stable, covalent manner. For cross-linking the antigen binding sites of the present invention methods known in the art can be used. For instance, these methods may involve the use of agents like MBS (m-Maleimidobenzoyl-N-hydroxysuccinimide ester), NHS(N-hydroxysuccinimide ester), EDC (1-ethyl-3-(3-dimethylaminopropyl) carbodiimide), glutaraldehyde, etc. These agents often require free functional groups (e.g. free SH-groups) in the molecules to be conjugated. In order to preserve the binding capacities of the antigen binding sites said binding sites should be ideally not affected by these agents.

“Complex” as used herein refers to at least two different antigen binding sites bound to each other in a non-covalent manner, e.g. by electrostatic, n-effects, van der Waals forces or hydrophobic effects.

The antibody construct of the present invention can also be a fusion protein. “Fusion protein” as used herein refers to a fusion of two or more amino acid sequences that are not naturally linked together. The fusion of the amino acid sequences can either be made by chemically linking ex vivo synthesized amino acid sequences. In an embodiment, the amino acid sequences are an in-frame translational fusion, i.e. correspond to a recombinant molecule expressed from a nucleic acid sequence in a prokaryotic or eukaryotic expression system. The term “fusion protein” as used herein refers also to a protein which may be created through genetic engineering from two or more proteins/peptides coding sequences joined together in a single polypeptide. In general, this is achieved by creating a “fusion gene”, a nucleic acid that encodes and expresses the fusion protein. For example, a fusion gene that encodes a fusion protein may be made by removing the stop codon from a first DNA sequence encoding the first protein, then appending a DNA sequence encoding the second protein in frame. The resulting fusion gene sequence will then be expressed by a cell as a single fusion protein. Fusion proteins may include a linker (or “spacer”) sequence which can promote appropriate folding and activity of each domain of the fusion protein. Fusion proteins may also include epitope tags for identification (e.g., in western blots, immunofluorescence, etc.) and/or purification. Non-limiting examples of epitope tags in current use include: HA-tag, myc-tag, FLAG-tag, HIS-tag (e.g. 6-HIS) and E-tag.

“Antigen binding site” or “binding site” as used herein denotes a region of an antigen binding molecule, preferably of a protein or a polypeptide, according to the invention to which a ligand (e.g. the antigen or antigen fragment of it) binds. The antigen binding site can have an artificial amino acid sequence obtainable by screening peptide libraries for peptides capable to bind to an antigen or may be derived from an antibody molecule or a fragment thereof. An antigen binding site can also be obtained from molecules like immunoglobulins. An antigen binding site is considered to specifically bind an antigen when the dissociation constant is preferably less than 1 μM, preferably less than 100 nM, more preferably less than 10 nM, even more preferably less than 1 nM, most preferably less than 100 μM.

As used herein, the term “specific for” can be used interchangeably with “binding to” or “binding specifically to”. “Specific for” refers to molecules that bind to an antigen or a group of antigens with greater affinity (as determined by, e.g., ELISA or BIAcore assays) than other antigens or groups of antigens. According to the present invention molecules “specific for” an antigen may also be able to bind to more than one, preferably more than two, more preferably more than three, even more preferably more than five, antigens. Such molecules are defined to be “cross-reactive” (e.g. cross-reactive immunoglobulins, cross-reactive antigen binding sites). For example, molecules being “specific for” the birch pollen allergen Bet v 1 may also bind to the major allergens of alder, hazel etc.

The antibody construct of the present invention can be a complex, conjugate or fusion protein comprising an antigen binding site specific for ICAM1 and antigen binding site specific for an allergen. The antigen binding sites can be part of one or more immunoglobulins or fragments thereof. Thus, according to a preferred embodiment of the present invention the antibody construct is a complex, conjugate or fusion protein comprising an immunoglobulin or an immunoglobulin fragment specific for ICAM1 and an immunoglobulin or an immunoglobulin fragment specific for an allergen.

The antigen binding site of the antibody construct of the present invention may be part of or derived from an immunoglobulin (i.e. antibody). Therefore, the antibody construct of the present invention can be a complex, conjugate or fusion protein as defined herein comprising an immunoglobulin or an immunoglobulin fragment specific for ICAM1 and an immunoglobulin or an immunoglobulin fragment specific for an allergen. Exemplary immunoglobulins/antibodies or immunoglobulin/antibody fragments are selected from the group consisting of polyclonal antibodies, monoclonal antibodies, chimeric antibodies, humanized antibodies, human antibodies, multispecific antibodies, Fab, Fab′, F(ab′)2, Fv, domain antibody (dAb), complementarity determining region (CDR) fragments, CDR-grafted antibodies, single-chain antibodies (ScFv), single chain antibody fragments, chimeric antibodies, diabodies, triabodies, tetrabodies, minibodies, linear antibodies, chelating recombinant antibodies, tribodies, bibodies, intrabodies, nanobodies, small modular immunopharmaceuticals (SMIP), camelized antibodies, VHH containing antibodies and polypeptides that comprise at least a portion of an immunoglobulin that is sufficient to confer specific antigen binding to the polypeptide, such as one, two, three, four, five or six CDR sequences. In a particularly preferred embodiment of the present invention the immunoglobulin is a single-chain antibody.

It is particularly preferred that the antigen binding site or the immunoglobulin or fragments thereof comprising said site are recombinant molecules.

Methods of generating antibodies and antibody fragments are well known in the art. For example, antibodies may be generated via any one of several methods which employ induction of in vivo production of antibody molecules, screening of immunoglobulin libraries or generation of monoclonal antibody molecules by cell lines in culture. These include, but are not limited to, the hybridoma technique, the human B-cell hybridoma technique and the Epstein-Barr virus (EBV)-hybridoma technique.

Monoclonal antibodies specific for ICAM1 or an allergen may be prepared by known techniques, for example those disclosed in “Monoclonal Antibodies: A manual of techniques”, H Zola (CRC Press, 1988). Antibody and immunoglobulin fragments can also be obtained using methods well known in the art (see, for example, Harlow and Lane, 1988, “Antibodies: A Laboratory Manual, Cold Spring Harbor Laboratory, New York). For instance, immunoglobulin fragments comprising the antigen binding sites of the present invention can be prepared by proteolytic hydrolysis of antibodies or by recombinant expression in eukaryotes or prokaryotes such as E. coli, mammalian (e.g. CHO cells), insect cells or yeast cells (e.g. P. pastoris) of DNA encoding the fragment comprising said antigen binding sites. Alternatively, antibody fragments can be obtained by pepsin or papain digestion of whole antibodies by conventional methods.

Preferably, the antibody or antigen-binding fragment thereof is a human antibody or humanized antibody. For human therapy humanized antibodies may be used. Humanized forms of non-human (e.g. murine) antibodies are genetically engineered chimeric antibodies or antibody fragments having minimal-portions derived from non-human antibodies. Humanized antibodies include antibodies in which complementarity determining regions of a human antibody (recipient antibody) are replaced by residues from a complementarity determining region of a non-human species (donor antibody) such as mouse, rat or rabbit having the desired functionality. In some instances, Fv framework residues of the human antibody are replaced by corresponding non-human residues. Humanized antibodies may also comprise residues which are found neither in the recipient antibody nor in the imported complementarity determining region or framework sequences. In general, humanized antibody will comprise substantially all or at least one, and typically two, variable domains, in which all or substantially all of the complementarity determining regions correspond to those of a non-human antibody and all, or substantially all, of the framework regions correspond to those of a relevant human consensus sequence. Humanized antibodies may also include at least a portion of an antibody constant region, such as an Fc region, typically derived from a human antibody. Methods for humanizing non-human antibodies are well known in the art. Generally, the humanized antibody has one or more amino acid residues introduced into it from a source which is non-human. Humanization can be performed, for instance, by substituting human complementarity determining regions with corresponding rodent complementarity determining regions. Accordingly, such humanized antibodies are chimeric antibodies, wherein substantially less than an intact human variable domain has been substituted by the corresponding sequence from a non-human species. In practice, humanized antibodies may be typically human antibodies in which some complementarity determining region residues and possibly some framework residues are substituted by residues from analogous sites in rodent antibodies.

Regardless of structure, an “immunoglobulin fragment” of the present invention binds with the same antigen that is recognized by the full-length immunoglobulin. For instance, antibody fragments may include isolated fragments consisting of the variable regions, such as the “Fv” fragments consisting of the variable regions of the heavy and light chains or recombinant single chain polypeptide molecules in which light and heavy variable regions are connected by a peptide linker (“ScFv proteins”). The term “fragment” refers to a portion of a polypeptide typically having at least 10 contiguous or at least 20 contiguous or at least 50 contiguous amino acids of the immunoglobulin. The “immunoglobulin fragment” has to consist at least of or to comprise at least an antigen binding site. Exemplary antigen binding fragments may be selected from the group consisting of Fv fragments (e.g. single chain Fv and disulphide-bonded Fv), and Fab-like fragments (e.g. Fab fragments, Fab′ fragments and F(ab)₂ fragments).

In one preferred embodiment of the present invention the antigen binding fragment is an ScFv. One advantage of using antibody fragments, rather than whole antibodies, is the simple and efficient recombinant expression of antigen binding fragments such as Fab, Fv, ScFv and dAb antibody fragments.

According to a preferred embodiment of the present invention the antibody construct of the present invention comprises an antigen binding site, an immunoglobulin or an immunoglobulin fragment specific for ICAM1 chemically cross-linked to an antigen binding site, an immunoglobulin or an immunoglobulin fragment specific for an allergen.

According to a particularly preferred embodiment of the present invention the antibody construct is a bispecific antibody, a bispecific diabody or a bispecific (Fab)₂ fragment, whereby a bispecific diabody comprising two single chain variable fragments (ScFv) is most preferred.

Bispecific antibodies, diabodies and (Fab)₂ fragments can be obtained by methods known in the art (see e.g. Chames and Baty Curr Opin Drug Disc Dev 12(2009):276).

Exemplary bispecific molecules of the invention comprise a single antibody that has two arms comprising different antigen binding sites, a single antibody that has one antigen-binding region or arm specific to ICAM1 and a second chain or arm specific to an allergen, a single chain antibody that has specificity to ICAM1 and an allergen, e.g., via two ScFvs linked in tandem by a (peptide) linker; a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)), a chemically-linked bispecific (Fab′)2 fragment, a Tandab, which is a fusion of two single chain diabodies resulting in a tetravalent bispecific antibody that has two binding sites for each of the target antigens, a flexibody, which is a combination of ScFvs with a diabody resulting in a multivalent molecule, a so called “dock and lock” molecule, based on the “dimerization and docking domain” in Protein Kinase A, which, when applied to Fabs, can yield a trivalent bispecific binding protein consisting of two identical Fab fragments linked to a different Fab fragment, a so-called Scorpion molecule, comprising, e.g., two ScFvs fused to both termini of a human Fab-arm, and a diabody.

Methods for preparing bispecific antibodies are known to the person skilled in the art (see e.g. WO 2008/119353, WO 2011/131746). Examples of other platforms useful for preparing bispecific antibodies include but are not limited to BITE (Micromet), DART (MacroGenics), Fcab and Mab2 (F-star) and Fc-engineered IgG₁ (Xencor). Of course, methods involving hybrid hybridoma and chemical conjugation (Marvin and Zhu Acta Pharmacol Sin 26(2005):649) can also be used. Co-expression in a host cell of two antibodies, consisting of different heavy and light chains, leads to a mixture of possible antibody products in addition to the desired bispecific antibody, which can then be isolated by, e.g., affinity chromatography or similar methods.

The antibody construct comprising antigen binding sites specific for ICAM1 and at least one allergen preferably comprises or consists of an intact antibody. Alternatively, the antibody construct comprises or consists of portions of intact antibodies sufficient to display binding specificity for ICAM1 and at least one allergen.

In an alternative embodiment, the antibody construct comprises or consists of antigen binding fragment selected from the group consisting of a Fv fragment, an Fab fragment, an Fab-like fragment and combinations thereof.

It is known in the art that ICAM1 is highly expressed on the respiratory epithelium of allergic patients and in the oral and gut epithelium. Therefore, it is particularly preferred to provide antibody constructs which comprise next to one or more antigen binding sites specific for ICAM1 one or more antigen binding sites specific for allergens which are characterized to be inhalation or food allergens.

“Inhalation allergens” are allergens which occur in nature in a form so that these allergens can be inhaled by a mammal or a human (e.g. pollen). Thus, “inhalation allergens” get usually in contact with respiratory epithelial cells present, for instance, in the lung and in tracheae and/or also with the surface of the eye.

“Food allergens”, as used herein, refers to allergens which are present in food ingested by a mammal or a human. “Food allergens”, thus, get in contact with the mouth (oral epithelium), with the esophagus and the gut (gut epithelium; gastrointestinal epithelium).

According to a particularly preferred embodiment of the present invention the inhalation allergen is selected from the group consisting of pollen allergens, mold allergens, mite allergens and animal allergens, preferably pet allergens.

According to a further embodiment of the present invention the allergen is selected from the group consisting of a pollen allergen, mite allergen, cat allergen, dog allergen, insect bite/sting allergen, legume allergen, fruit allergen, nut allergen, milk allergen and fish allergen.

The allergen to which the antigen binding site of the antibody construct of the present invention binds to or is specific for is preferably a pollen allergen selected from the group of a grass pollen allergen, preferably the grass pollen allergen Phl p 1, Phl p 2, Phl p 4, Phl p 5, Phl p 6, Phl p 7, Phl p 11, Phl p 12 or Phl p 13, a weed allergen, preferably the ragweed allergen Amb a 1, Amb a 2, Amb a 3, Amb a 4, Amb a 5, Amb a 6, Amb a 7, Amb a 8, Amb a 9 or Amb a 10, a mugwort allergen, preferably Art v 1, Art v 2, Art v 3, Art v 4, Art v 5 or Art v 6, a Paritaria pollen allergen, preferably Par j 1, Par j 2, Par j 3 or Par j 4, an olive pollen allergen, preferably Ole e 1, Ole e 2, Ole e 3, Ole e 4, Ole e 5, Ole e 6, Ole e 7, Ole e 8, Ole e 9, Ole e 10 or Ole e 11, a pollen allergen from Saltwort thistle, preferably Sal k 1, Sal k 2, Sal k 3, Sal k 4 or Sal k 5, and a tree pollen, preferably a Cypress pollen, preferably Cha o 1 or Cha o 2, or a cedar pollen, preferably Jun a 1, Jun a 2 or Jun a 3, or a birch pollen allergen, preferably Bet v 1, whereby Phl p 1, Phl p 2, Phl p 5 and Bet v 1 are most preferred.

According to a particularly preferred embodiment of the present invention the allergen is Phl p 2 or Phl p 5 so that the antigen binding site of the antibody construct is specific for and binds to Phl p 2 and Phl p 5, respectively.

According to a preferred embodiment of the present invention the antibody construct comprises an antigen binding site or immunoglobulin or immunoglobulin fragment specific for Phl p 2, comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7 and combinations thereof.

SEQ ID NO: 1:
LESGPGLVKPAQTLSLSCAVSGGSIRSGGYYWSWIRQHPGKGLEWIGYIY
HSGNTYYNPSLKSRIAMSVDTSENKFSLRLNSVTAADTAVYYCARLDGYT
LDIWGQGTLVTVSS
SEQ ID NO: 2:
ELTQSPSSLSASVGDRVTISCRASQRINTYLNWYQHKPGKAPKLLIYAAS
SLQSGVPSRFSGSGYGTDFTLTISSLQPEDFASYYCQESLSASYTFGQGT
KVEIKR
SEQ ID NO: 3:
MAQVQLQESGPGLVKPAQTLSLSCAVSGGSIRSGGYYWSWIRQHPGKGLE
WIGYIYHSGNTYYNPSLKSRIAMSVDTSENKFSLRLNSVTAADTAVYYCA
RLDGYTLDIWGQGTLVTVSSGGGGSGGGGSGGGGSETTLTQSPSSLSASV
GDRVTISCRASQRINTYLNWYQHKPGKAPKLLIYAASSLQSGVPSRFSGS
GYGTDFTLTISSLQPEDFASYYCQESLSASYTFGQGTKVEIKRAAAGAPV
PYPDPLEPR
SEQ ID NO: 4:
LESGPGLVKPSQTLSLTCTVSGGSIRSGGYYWSWVRQPPGKGLEWIGNIY
HSGNTYYNPSLKSRITMSVDTSKNHFSLRLTSVTAADTAVYYCARSDGYT
LDNWGQGTLVTVSS
SEQ ID NO: 5:
ELTQSPSSLSASVGDRVTITCRARQSISTYLNWYQQKPGKAPKLLIWSAS
NLQSGVPSRFSGSGSGTEFTLTISNLQPEDFASYYCQQSYTTLYTFGPGT
KLEIKR
SEQ ID NO: 6:
LESGPGLVKPSQTLSLTCTVSGGSIRSGGYYWSWIRQPPGKGLEWIGYIY
HSGNTYYNPSLKSRVTMSVDTSKNHFSLRLSSVTAADTAVYYCARSDGYT
LDNWGQGTLVTVSS
SEQ ID NO: 7:
ELTQSPSSVSASVGDRVTITCRASQGISSWLAWYQQKPGKAPKLLIYSAS
SLQSGVPSRFSGSGSGTDFSLTISSLQPEDSATYYCQQANSFPYTFGQGT
KVEIKR

The antibody construct of the present invention able to bind to Phl p 2 comprises preferably SEQ ID NO: 1 and 2, SEQ ID NO: 3, SEQ ID NO: 4 and 5, SEQ ID NO: 6 and 7, or combinations thereof.

According to a preferred embodiment of the present invention the immunoglobulin specific for Phl p 2 comprises at least one CDR sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19. The immunoglobulin specific for Phl p 2 comprises preferably CDR sequences SEQ ID NO: 8, SEQ ID NO: 9 and SEQ ID NO: 10; and/or SEQ ID NO: 11, SEQ ID NO: 12 and SEQ ID NO: 13; and/or SEQ ID NO: 8, SEQ ID NO: 9 and SEQ ID NO: 14; and/or SEQ ID NO: 15, SEQ ID NO: 16 and SEQ ID NO: 17; and/or SEQ ID NO: 18, SEQ ID NO: 16 and SEQ ID NO: 19. The immunoglobulin specific for Phl p 2 comprises preferably a heavy chain comprising CDR sequences SEQ ID NO: 8, SEQ ID NO: 9 and SEQ ID NO: 10; and a light chain comprising CDR sequences SEQ ID NO: 11, SEQ ID NO: 12 and SEQ ID NO: 13; a heavy chain comprising CDR sequences SEQ ID NO: 8, SEQ ID NO: 9 and SEQ ID NO: 14; and a light chain comprising CDR sequences SEQ ID NO: 15, SEQ ID NO: 16 and SEQ ID NO: 17; or a heavy chain comprising CDR sequences SEQ ID NO: 8, SEQ ID NO: 9 and SEQ ID NO: 14; and a light chain comprising CDR sequences SEQ ID NO: 18, SEQ ID NO: 16 and SEQ ID NO: 19 (see Table A below).

According to a further preferred embodiment of the present invention the antibody construct comprises an antigen binding site or immunoglobulin or immunoglobulin fragment specific for Phl p 1, comprising an amino acid sequence selected from the group consisting of SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and combinations thereof.

SEQ ID NO: 20:
LESGGGWVQPGRSLRLSCAASGFTFDDHAMHWVRQAPGKGLEWVAGVSWN
SGTRAYADSVKGRFTISRDNAKNSLYLEMKSLRAEDTAIYYCARDMRAAT
PYSFDYWGQGVLVTVSS
SEQ ID NO: 21:
ELVMTQSPSSLSASVGDRVTITCRASQTFNNYLNWYQQKPGKAPNLLIYA
ASTLRRGIPSRFSGSGSGTYFTLTINGLQPEDFATYYCQQSYSTPLTFGG
GTKVDIKR
SEQ ID NO: 22:
LESGGGLVQPGGSLRLSCAASGFTFDDHAMHWVRQTPEKGLQWVSGISWN
SGVVAYADSVKGRFSISRDNAKNSLYLQMNNLIPEDTAVYYCVRDFRAAS
PYFFDYWGQGTRVTVSS
SEQ ID NO: 23:
ELTQSPSSLSASVGDRVTITCRASQSIGNYLNWYQQKPGKAPNLLIYAAS
SLQSGVPSRFSGSGSGTDFTLTISSLQREDFATYYCQQSNRTPITFGQGT
RLEIKG
SEQ ID NO: 24:
MAQVNLRESGGGLVQPGGSLRLSCAASGFTFDDHAMHWVRQTPEKGLQWV
SGISWNSGVVAYADSVKGRFSISRDNAKNSLYLQMNNLIPEDTAVYYCVR
DFRAATPYFFDYWGQGTLVTVSSGGGGSGGGGSGGGGSETTLTQSPSSLS
ASVGDRVTITCRASQSIGNYLNWYQQKPGKAPNLLIYAASSLQSGVPSRF
SGSGSGTDFTLTISSLQREDFATYYCQQSNRTPITFGQGTRLEIKRAAAG
APVPYPDPLEPR
SEQ ID NO: 25:
LESGGGWVQPGRSLRLSCSASGLTFDDYAMHWVRQAPGKGLEWVSGISWN
SGVRAYADSVKGRFTISRDNGKNSLYLQMNSLRPEDTALYYCAKDIRAAT
PYALDHWGQGVLVTVSS
SEQ ID NO: 26:
ELVMTQSPSSLSASEGDRVTITCRASRTIYNYLNWYQQKPGRAPKLLIHA
ASTLQDGVPSRFSGSGSGTEFTLTISGLQSEDFATYYCQQSHGTPLTFGG
GTTVDVKR
SEQ ID NO: 27:
MAQVQLQESGGGLVQPGGSLKLSCAASEFTFNNYWMSWVRQAPGKGLEWV
ANIKPDGSVKYYVDSVKGRFTISRDNAKNSLFLQMNSLRAEDTAVYFCAR
ERQSGAFGLWGQGTTVTVSSGAGGSGGGGSGGGGSDIQMTQSPSSLSASV
GDRVTITCRASQSISSYLNWYQQKPGKAPKLLIYAASSLQSGVPSRFSGS
GSGTDFTLTISSLQPEDFATYYCQQSYSTPLTFGGGTKVEIKRAAAGAPV
PYPDPLEPR
SEQ ID NO: 28:
MAQVNLRESGGGLVRPGRSLRLSCAASGFTFDDYAMHWVRQAPGKGLEWV
SGISWNSGSIGYADSVKGRFTISRDNAKNSLYLQMNSLRAEDTAVYYCAR
EWDGTAYYGYTVGYWGQGTLVTVSSGGGGSGGGGSGGGGSDVVMTQSPLS
LPVTLGQPASISCRSSQSLVYSDGNTYLNWFQQRPGQSPRRLIYKVSNRD
SGVPDRFSGSGSGTDFTLKISRVEAEDVGVYYCMQGTHWPPTFGQGTRLE
IKRAAAGAPVPYPDPLEPR
SEQ ID NO: 29:
MAQVQLQESGGGVVQPGRSLRLSCAASGFTFSSYAMSWVRQAPGKGLEWV
SGISGSGGSTYYADSVKGRFTISRDNSKNTLYLQMNSLRAEDTAIYYCAR
VLRNQAAAGTIRWFDPWGQGTLVTVSSGGGGSGGGGSGGGGSDVVMTQSP
LSLPVTLGQPASISCRSSQSLVYSDGNTYLNWFQQRPGQSPRRLIYKVSN
RDSGVPDRFSGSGSGTDFTLKIRRVEAEDVGVYYCMQGTHWPPTFGQGTK
LEIKRAAAGAPVPYPDPLEPR

The antibody construct of the present invention able to bind to Phl p 1 comprises preferably SEQ ID NO: 20 and 21, SEQ ID NO: 22 and 23, SEQ ID NO: 24, SEQ ID NO: 25 and 26, SEQ ID NO: 27, SEQ ID NO: 28, SEQ ID NO: 29 or combinations thereof.

According to a preferred embodiment of the present invention the immunoglobulin specific for Phl p 1 comprises at least one CDR sequence selected from the group consisting of SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56 and SEQ ID NO: 57. The immunoglobulin specific for Phl p 1 comprises preferably CDR sequences SEQ ID NO: 30, SEQ ID NO: 31 and SEQ ID NO: 32; and/or SEQ ID NO: 33, SEQ ID NO: 12 and SEQ ID NO: 34; and/or SEQ ID NO: 30, SEQ ID NO: 35 and SEQ ID NO: 36; and/or SEQ ID NO: 37, SEQ ID NO: 12 and SEQ ID NO: 38; and/or SEQ ID NO: 39, SEQ ID NO: 40 and SEQ ID NO: 41; and/or SEQ ID NO: 42, SEQ ID NO: 12 and SEQ ID NO: 43; and/or SEQ ID NO: 44, SEQ ID NO: 45 and SEQ ID NO: 46; and/or SEQ ID NO: 47, SEQ ID NO: 12 and SEQ ID NO: 34; and/or SEQ ID NO: 48, SEQ ID NO: 49 and SEQ ID NO: 50; and/or SEQ ID NO: 51, SEQ ID NO: 52 and SEQ ID NO: 53; and/or SEQ ID NO: 54, SEQ ID NO: 55 and SEQ ID NO: 56; and/or SEQ ID NO: 57, SEQ ID NO: 52 and SEQ ID NO: 53. The immunoglobulin specific for Phl p 1 comprises preferably a heavy chain comprising CDR sequences SEQ ID NO: 30, SEQ ID NO: 31 and SEQ ID NO: 32; and a light chain comprising CDR sequences SEQ ID NO: 33, SEQ ID NO: 12 and SEQ ID NO: 34; a heavy chain comprising CDR sequences SEQ ID NO: 30, SEQ ID NO: 35 and SEQ ID NO: 36; and a light chain comprising CDR sequences SEQ ID NO: 37, SEQ ID NO: 12 and SEQ ID NO: 38; or a heavy chain comprising CDR sequences SEQ ID NO: 39, SEQ ID NO: 40 and SEQ ID NO: 41; and a light chain comprising CDR sequences SEQ ID NO: 42, SEQ ID NO: 12 and SEQ ID NO: 43; or a heavy chain comprising CDR sequences SEQ ID NO: 44, SEQ ID NO: 45 and SEQ ID NO: 46; and a light chain comprising CDR sequences SEQ ID NO: 47, SEQ ID NO: 12 and SEQ ID NO: 34; or a heavy chain comprising CDR sequences SEQ ID NO: 48, SEQ ID NO: 49 and SEQ ID NO: 50; and a light chain comprising CDR sequences SEQ ID NO: 51, SEQ ID NO: 52 and SEQ ID NO: 53; or a heavy chain comprising CDR sequences SEQ ID NO: 54, SEQ ID NO: 55 and SEQ ID NO: 56; and a light chain comprising CDR sequences SEQ ID NO: 57, SEQ ID NO: 52 and SEQ ID NO: 53 (see Table A below).

According to a further preferred embodiment of the present invention the antibody construct comprises an antigen binding site or immunoglobulin or immunoglobulin fragment specific for Phl p 5, comprising an amino acid sequence selected from the group consisting of SEQ ID NO: 72, SEQ ID NO: 73, SEQ ID NO: 74, SEQ ID NO: 75, SEQ ID NO: 76 and combinations thereof.

SEQ ID NO: 72:
LESGGGVVQPGRSLRLSCAASEFAFDSYEIYWVRQAPGKGLEWVAMISID
ETNKHYADSVKGRFTIFRDNSNKTVHLQMNNLRSEDSGVYYCARWNNWNH
RGMVFAFDLWGQGTMVTVSS
SEQ ID NO: 73:
ELTQSPSSVSASVGDRVTITCRASQGISSWLAWYQQKPGKAPKLLIYSAS
SLQSGVPSRFSGSGSGTDFSLTISSLQPEDSATYYCQQANSFPYTFGQGT
KVEIKR
SEQ ID NO: 74:
ELVMTQSPSSVSASVGDRVTITCRASHSISNYLNWYQQKPGKAPKLLIYA
ASSLQSGVPSRFSGSGSGTDFTLTISSLQPEDFANYYCQESFSPSGTFGQ
GTKVEIKR
SEQ ID NO: 75:
ELTQSPSSLSASVGDRVTITCRASQSILGYLNWYQQKPGKAPKLLIYAAS
TLQSGVPSRFSGSGSGTDFTLTITSLQPDDFATYYCQQSYITPRTFGQGT
KVEVKR
SEQ ID NO: 76:
ELVMTQSPSSLSAFVGDRVTITCRASQSISSYLNWYQQKPGKAPKLLIYA
ASSLQSGVPSRFSGSGSGTDFTLTISSLQPEDFATYYCQQSHSTPYTFGQ
GTNLEIKR

The antibody construct of the present invention able to bind to Phl p 5 comprises preferably SEQ ID NO: 72 and 73, SEQ ID NO: 74, SEQ ID NO: 75, SEQ ID NO: 76 or combinations thereof.

According to a preferred embodiment of the present invention the immunoglobulin specific for Phl p 5 comprises at least one CDR sequence selected from the group consisting of SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 12, SEQ ID NO: 16, SEQ ID NO: 19 and SEQ ID NO: 47. The immunoglobulin specific for Phl p 5 comprises preferably CDR sequences SEQ ID NO: 77, SEQ ID NO: 78 and SEQ ID NO: 79; SEQ ID NO: 80, SEQ ID NO: 16 and SEQ ID NO: 19; and/or SEQ ID NO: 81, SEQ ID NO: 12 and SEQ ID NO: 82; and/or SEQ ID NO: 83, SEQ ID NO: 12 and SEQ ID NO: 84; and/or SEQ ID NO: 47, SEQ ID NO: 12 and SEQ ID NO: 85. The immunoglobulin specific for Phl p 5 comprises preferably a heavy chain comprising CDR sequences SEQ ID NO: 77, SEQ ID NO: 78 and SEQ ID NO: 79; and a light chain comprising CDR sequences SEQ ID NO: 80, SEQ ID NO: 16 and SEQ ID NO: 19; or a light chain comprising CDR sequences SEQ ID NO: 81, SEQ ID NO: 12 and SEQ ID NO: 82; or a light chain comprising CDR sequences SEQ ID NO: 83, SEQ ID NO: 12 and SEQ ID NO: 84; or a light chain comprising CDR sequences SEQ ID NO: 47, SEQ ID NO: 12 and SEQ ID NO: 85.

According to a further preferred embodiment of the present invention the antibody construct comprises an antigen binding site or immunoglobulin or immunoglobulin fragment specific for Phl p 6, comprising an amino acid sequence selected from the group consisting of SEQ ID NO: 86, SEQ ID NO: 87, SEQ ID NO: 88 and combinations thereof.

SEQ ID NO: 86:
EVQLVQSGGGLVKPGGSLRLSCAASGFIFSDHYMAWVRQTPGKGLEWISY
ISMTSSYTNYADSVKGRFTISRDNAKNSLSLEMNSLRVEDTAVYYCARDR
LRNWNDDAFDIWGPGTMVTVSSGGGGSGGGGSGGGGSSYELTQPASVSGS
PGQSITISCTGTPSDIGTYPYVSWYQQHPDRAPKLLIYGVTNRPSGVSNR
FSGSKSGNTASLTISELQAEDEADYYCSSYTLTSIVVFGGGTKLTVLGDY
KDDDDK
SEQ ID NO: 87:
EVQLVQSGGGLVKPGGSLRLSCAASGFIFSDHYMAWVRQTPGKGLEWISY
ISMTSSYTNYADSVKGRFTISRDNAKNSLSLEMNSLRVEDTAVYYCARDR
LRNWNDDAFDIWGPGTMVTVSS
SEQ ID NO: 88:
SYELTQPASVSGSPGQSITISCTGTPSDIGTYPYVSWYQQHPDRAPKLLI
YGVTNRPSGVSNRFSGSKSGNTASLTISELQAEDEADYYCSSYTLTSIVV
FGGGTKLTVLGDYKDDDDK

The antibody construct of the present invention able to bind to Phl p 6 comprises preferably SEQ ID NO: 86, SEQ ID NO: 87 and SEQ ID NO: 88 or combinations thereof.

According to a preferred embodiment of the present invention the immunoglobulin specific for Phl p 6 comprises at least one CDR sequence selected from the group consisting of SEQ ID NO: 89, SEQ ID NO: 90, SEQ ID NO: 91, SEQ ID NO: 92, SEQ ID NO: 93 and SEQ ID NO: 94. The immunoglobulin specific for Phl p 6 comprises preferably CDR sequences SEQ ID NO: 89, SEQ ID NO: 90 and SEQ ID NO: 91; and/or SEQ ID NO: 92, SEQ ID NO: 93 and SEQ ID NO: 94. The immunoglobulin specific for Phl p 6 comprises preferably a heavy chain comprising CDR sequences SEQ ID NO: 89, SEQ ID NO: 90 and SEQ ID NO: 91; and a light chain comprising CDR sequences SEQ ID NO: 92, SEQ ID NO: 93 and SEQ ID NO: 94.

According to a particularly preferred embodiment of the present invention the allergen is Bet v 1 so that the antigen binding site of the antibody construct is specific for and binds to Bet v 1.

According to a preferred embodiment of the present invention the antibody construct comprises an antigen binding site or immunoglobulin or immunoglobulin fragment specific for Bet v 1, preferably comprising an amino acid sequence selected from the group consisting of SEQ ID NO: 58, SEQ ID NO: 65 and SEQ ID NO: 66.

SEQ ID NO: 58:
MAQVQLVQSGAEVKKPGSSVKVSCKASGGTFSSYAISWVRQAPGQGLEWM
GRIIPILGIVNYAQKFQGRVTITADKSTSTAYMELSSLRSEDTAVYYCAR
EPLGYCSGGSCYPLHNGMDVWGQGTTVTVSSSGGGSGGGGSGGGGSDIQM
TQSPDSLAVSLGERATINCKSSQSVLYSSNNKNYLAWYQQKPGQPPKLLF
YWASTRESGVPDRFSGSGSGTDFTLTISSLQAEDVAVYYCQQYYTTPLTF
GQGTKLEIKRAAAGAPVPYPDPLEPR
SEQ ID NO: 65:
MEFGLSWLFLVAFLKGVQCEVQLLESGGGLVQPGGSLRLSCVASGFTFTN
YAMTWVRQAPGKGLEWVSAISNYGGSTYYSDSVKGRFTISRDNSKSTLSL
HMNSLRAEDTALYYCAKYFDFWGQGTLVTVSS
SEQ ID NO: 66:
MRLPAQLLGLLLLWVPGAGCDIQLTQSPSSLSASVGDRVTITCRVSQGIS
NCLNWYRRKPGKVNKVLFYSASNFQSGVPSRFSASASGTDLTLSVNTLQP
EDVATCYCQRTDNALHHFAQGTRLEIKR

The antibody construct of the present invention able to bind to Bet v 1 comprises preferably SEQ ID NO: 58 or SEQ ID NO: 65 and SEQ ID NO: 66.

According to a preferred embodiment of the present invention the immunoglobulin specific for Bet v 1 comprises at least one CDR sequence selected from the group consisting of SEQ ID NO: 59, SEQ ID NO: 60, SEQ ID NO: 61, SEQ ID NO: 62, SEQ ID NO: 63, SEQ ID NO: 64, SEQ ID NO: 67, SEQ ID NO: 68, SEQ ID NO: 69, SEQ ID NO: 70, SEQ ID NO: 16 and SEQ ID NO: 71. The immunoglobulin specific for Bet v 1 comprises preferably CDR sequences SEQ ID NO: 59, SEQ ID NO: 60 and SEQ ID NO: 61; and/or SEQ ID NO: 62, SEQ ID NO: 63 and SEQ ID NO: 64; and/or SEQ ID NO: 67, SEQ ID NO: 68 and SEQ ID NO: 69; and/or SEQ ID NO: 70, SEQ ID NO: 16 and SEQ ID NO: 71. The immunoglobulin specific for Bet v 1 comprises preferably a heavy chain comprising CDR sequences SEQ ID NO: 59, SEQ ID NO: 60 and SEQ ID NO: 61; and a light chain comprising CDR sequences SEQ ID NO: 62, SEQ ID NO: 63 and SEQ ID NO: 64; or a heavy chain comprising CDR sequences SEQ ID NO: 67, SEQ ID NO: 68 and SEQ ID NO: 69; and a light chain comprising CDR sequences SEQ ID NO: 70, SEQ ID NO: 16 and SEQ ID NO: 71.

TABLE A

| SEQ | | |
|---|---|---|
| ID | | |
| NO: | Sequence | CDR |
| 8 | GGSIRSGGYY | CDR1 |
| 9 | IYHSGNT | CDR2 |
| 10 | ARLDGYTLDIW | CDR3 |
| 11 | QRINTY | CDR1 |
| 12 | AAS | CDR2 |
| 13 | QESLSASYT | CDR3 |
| 14 | ARSDGYTLDN | CDR3 |
| 15 | QSISTY | CDR1 |
| 16 | SAS | CDR2 |
| 17 | QQSYTTLYT | CDR3 |
| 18 | QGISSY | CDR1 |
| 19 | QQANSFPYT | CDR3 |
| 30 | GFTFDDHA | CDR1 |
| 31 | VSWNSGTR | CDR2 |
| 32 | ARDMRAATPYSFDY | CDR3 |
| 33 | QTFNNY | CDR1 |
| 34 | QQSYSTPLT | CDR3 |
| 35 | ISWNSGVV | CDR2 |
| 36 | VRDFRAASPYFFDY | CDR3 |
| 37 | QSIGNY | CDR1 |
| 38 | QQSNRTPIT | CDR2 |
| 39 | GLTFDDYA | CDR1 |
| 40 | ISWNSGVR | CDR2 |
| 41 | AKDIRAATPYALDH | CDR3 |
| 42 | RTIYNY | CDR1 |
| 43 | QQSHGTPLT | CDR3 |
| 44 | EFTFNNYW | CDR1 |
| 45 | IKPDGSVK | CDR2 |
| 46 | ARERQSGAFGL | CDR3 |
| 47 | QSISSY | CDR1 |
| 48 | GFTFDDYA | CDR1 |
| 49 | ISWNAGSI | CDR2 |
| 50 | AREWDGTAYYGYTVGY | CDR3 |
| 51 | QSLVYSDGNTY | CDR1 |
| 52 | KVS | CDR2 |
| 53 | MQGTHWPPT | CDR3 |
| 54 | GFTFSSYA | CDR1 |
| 55 | ISGSGGST | CDR2 |
| 56 | ARVLRNQAAAGTIRWEDP | CDR3 |
| 57 | QSLVYSDGNT | CDR1 |
| 59 | GGTFSSYA | CDR1 |
| 60 | IIPILGIV | CDR2 |
| 61 | AREPLGYCSGGSCYPLHNGMDV | CDR3 |
| 62 | QSVLYSSNNKNY | CDR1 |
| 63 | WAS | CDR2 |
| 64 | QQYYTTPLT | CDR3 |
| 67 | GFTFTNYA | CDR1 |
| 68 | ISNYGGST | CDR2 |
| 69 | AKYFDF | CDR3 |
| 70 | QGISNC | CDR1 |
| 71 | QRTDNALHH | CDR3 |
| 77 | EFAFDSYE | CDR1 |
| 78 | ISIDETNK | CDR2 |
| 79 | ARWNNWNHRGMVFAFDL | CDR3 |
| 80 | QGISSW | CDR1 |
| 81 | HSISNY | CDR1 |
| 82 | QESFSPSGT | CDR3 |
| 83 | QSILGY | CDR1 |
| 84 | QQSYITPRT | CDR3 |
| 85 | QQSHSTPYT | CDR3 |
| 89 | GFIFSDHY | CDR1 |
| 90 | ISMTSSYT | CDR2 |
| 91 | ARDRLRNWNDDAFDI | CDR3 |
| 92 | PSDIGTYPY | CDR1 |
| 93 | GVT | CDR2 |
| 94 | SSYTLTSIVV | CDR3 |

According to another preferred embodiment of the present invention the allergen is a mite allergen, preferably Der p 1, Der p 2, Der p 3, Der p 4, Der p 5, Der p 6, Der p 7, Der p 8, Der p 9, Der p 10, Der p 11, Der p 14, Der p 15, Der p 18, Der p 20, Der p 21 and Der p 23.

According to a further preferred embodiment of the present invention the allergen is an animal, allergen, selected from a group consisting of a cat allergen, preferably Fel d 1, Fel d 2, Fel d 3, Fel d 4, Fel d 5, Fel d 6, Fel d 7 and Fel d 8, a dog allergen, preferably Can f 1, Can f 2, Can f 3, Can f 4 and Can f 5, and a cockroach allergen, preferably Bla g 1, Bla g 2, Blag 3, Blag 4, Blag 5, Blag 6, Bla g 7, Bla g 8, Bla g 11, Per a 1, Per a 2, Per a 3, Per a 6, Per a 7, Per a 9 and Per a 10.

According to preferred embodiment of the present invention the immunoglobulin specific for ICAM1 is an ICAM1-specific human or humanized mammalian, preferably mouse, IgG₁ antibody.

The antigen binding site specific for or binding to ICAM1 can be part of an immunoglobulin which has been obtained by methods known in the art. Particularly preferred the immunoglobulin or immunoglobulin fragment comprising said antigen binding site is an IgG, more preferably an IgG₁, antibody or fragment thereof.

Another aspect of the present invention relates to a pharmaceutical composition comprising the antibody construct of the present invention.

The antibody construct of the present invention can be provided in a pharmaceutical composition which may comprise next to at least one antibody construct of the present invention further ingredients such as other active compounds and/or at least one pharmaceutically acceptable excipient.

In a particularly preferred embodiment the pharmaceutical composition of the present invention comprises an antibody construct as defined above which comprises an antigen binding site or immunoglobulin or immunoglobulin fragment specific for Bet v 1 and/or for Phl p 1, Phl p 2, Phl p 5, Phl p 6 or combinations thereof.

“Pharmaceutically acceptable excipient” as used herein refers to a carrier, usually a liquid, in which an active therapeutic agent is formulated. The excipient generally does not provide any pharmacological activity to the formulation, though it may provide chemical and/or biological stability, and release characteristics. Exemplary formulations can be found, for example, in Remington's Pharmaceutical Sciences, 19th Ed., Grennaro, A., Ed., 1995. The term includes further any and all solvents, dispersion media, coatings, antibacterial and antifungal agents, isotonic and absorption delaying agents that are physiologically compatible. In one embodiment, the carrier is suitable for intranasal, oral or ocular administration or for inhalation. Pharmaceutically acceptable carriers and excipients include sterile aqueous solutions or dispersions and sterile powders. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions. A pharmaceutical composition of the present invention which is adapted for oral administration comprises preferably an enteric coating to protect the pharmaceutically active compounds from the low pH levels present in the stomach.

Pharmaceutical compositions typically must be sterile and stable under the conditions of manufacture and storage. The invention contemplates that the pharmaceutical composition is present in lyophilized form. The composition can be formulated as a solution, microemulsion, liposome, or other ordered structure. The earner can be a solvent or dispersion medium containing, for example, water, polyol (for example, glycerol, propylene glycol, and liquid polyethylene glycol), and suitable mixtures thereof. The invention further contemplates the inclusion of a stabilizer in the pharmaceutical composition. The proper fluidity can be maintained, for example, by the use of a coating such as lecithin, by the maintenance of the required particle size in the case of dispersion and by the use of surfactants.

The pharmaceutical composition of the present invention comprises preferably isotonic agents such as, for example, sodium chloride. For each of the recited embodiments, the compounds can be administered by a variety of dosage forms. Any biologically-acceptable dosage form known to persons skilled in the art, and combinations thereof, are contemplated. Examples of such dosage forms include reconstitutable powders, liquids, solutions, suspensions, emulsions, powders, granules, particles, microparticles, cachets, inhalants, aerosol inhalants, particle inhalants, and combinations thereof.

According to a preferred embodiment of the present invention the composition is adapted for intranasal, oral or ocular administration or for inhalation.

According to a further preferred embodiment of the present invention the pharmaceutical composition of the present invention is used for treating and/or preventing an allergy.

According to another preferred embodiment of the present invention the pharmaceutical composition of the present invention is used for desensitizing an allergic patient or a patient at risk to develop an allergy.

The pharmaceutical composition of the present invention cannot only be used in the treatment and in the prevention of an allergy.

According to a preferred embodiment of the present invention the pharmaceutical composition of the present invention is used for preventing, reducing and/or alleviating allergic symptoms in a patient suffering from an allergy or allergic symptoms.

According to a further preferred embodiment of the present invention the pharmaceutical composition of the present invention comprises 0.05 to 1000 μg, preferably 5 to 1000 μg, more preferably 50 to 1000 μg, even more preferably 100 to 1000 μg, of the antibody construct of the present invention.

Administration of the antibody construct and the pharmaceutical preparations according to the present invention to an individual in need thereof can be carried out using known procedures at dosages and for periods required. Effective amounts of the therapeutic compositions will vary according to factors such as the age, sex and weight of the individual and the ability of the protein or fragment thereof to elicit an antigenic response in the individual. The antibody construct and the pharmaceutical preparations may be administered in a convenient manner such as by intranasal, oral or ocular administration or by inhalation, whereby for inhalation allergens the administration by inhalation is most preferred. For example, about 0.05 to 1000 μg, more preferably from about 0.1 to 100 μg, even more preferably 0.5 to 100 μg, of active compound (i.e. antibody construct) per dosage unit may be administered. Dosage regimen may be adjusted to provide the optimum therapeutic response. For example, several divided doses may be administered daily or the dose may be proportionally reduced as indicated by the exigencies of the therapeutic situation. The pharmaceutical preparation of the present invention can be administered, for instance, for 1 to 6 times before an allergy season at intervals of 1 to 5 weeks and/or during the allergy season. During an allergy season the pharmaceutical preparation of the present invention is preferably administered at intervals in between 6 and 96 hours, preferably between 12 and 48 hours. If the allergen is a food allergen, the antibody construct of the present invention can be administered before or during the uptake of the respective food. The administration occurs preferably 1 to 60 minutes, more preferably 2 to 40 minutes, even more preferably 5 to 30 minutes, before the allergen comprising food is consumed.

The terms “preventing” and “prevention”, as used herein, refer to the prevention or inhibition of the recurrence, onset and development of an allergy or a symptom thereof in a subject resulting from the administration of the fusion protein or pharmaceutical preparation according to the present invention. In some embodiments “preventing” and “prevention” refers to the reduction of the risk to develop an allergy against specific allergens. The term “preventing” covers measures not only to prevent the occurrence of an allergy, but also to arrest its progress and reduce its consequences once established.

The terms “treatment” and “treating”, as used herein, refer to the reduction or inhibition of the progression and duration of an allergy, the reduction or amelioration of the severity of the allergy and the amelioration of one or more symptoms thereof. “Treatment” encompasses also the improvement and/or reversal of the symptoms of an allergy or allergic reactions. A fusion protein which causes an improvement in any parameter associated with allergy may be identified as a therapeutic protein. The term “treatment” refers to both therapeutic treatment and prophylactic measures. For example, those who may benefit from treatment with compositions and methods of the present invention include those already with an allergy as well as those in which the allergy is to be prevented.

A further aspect of the present invention relates to a method of treating and/or preventing an allergy, or preventing, reducing and/or alleviating allergic symptoms in a patient suffering from an allergy or allergic symptoms. In a particularly preferred embodiment birch pollen and grass pollen allergy are treated with an antibody construct as defined above which comprise an antigen binding site or immunoglobulin or immunoglobulin fragment specific for Bet v 1 and/or for Phl p 1, Phl p 2, Phl p 5, Phl p 6 or combinations thereof.

The present invention is further illustrated in the following figures and examples, however, without being restricted thereto.

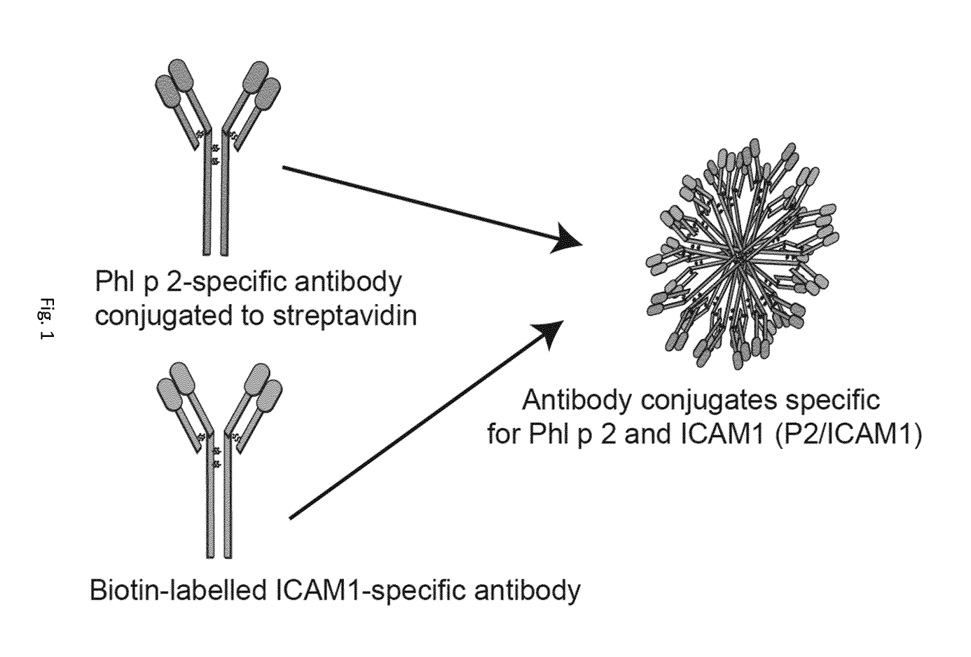

FIG. 1. Generation of antibody conjugates containing Phl p 2- and ICAM1-specific antibodies. A Phl p 2-specific monoclonal human IgG₁ antibody was conjugated to streptavidin and subsequently conjugates, termed P2/ICAM1 were formed with a biotin-conjugated ICAM1-specific mouse monoclonal antibody.

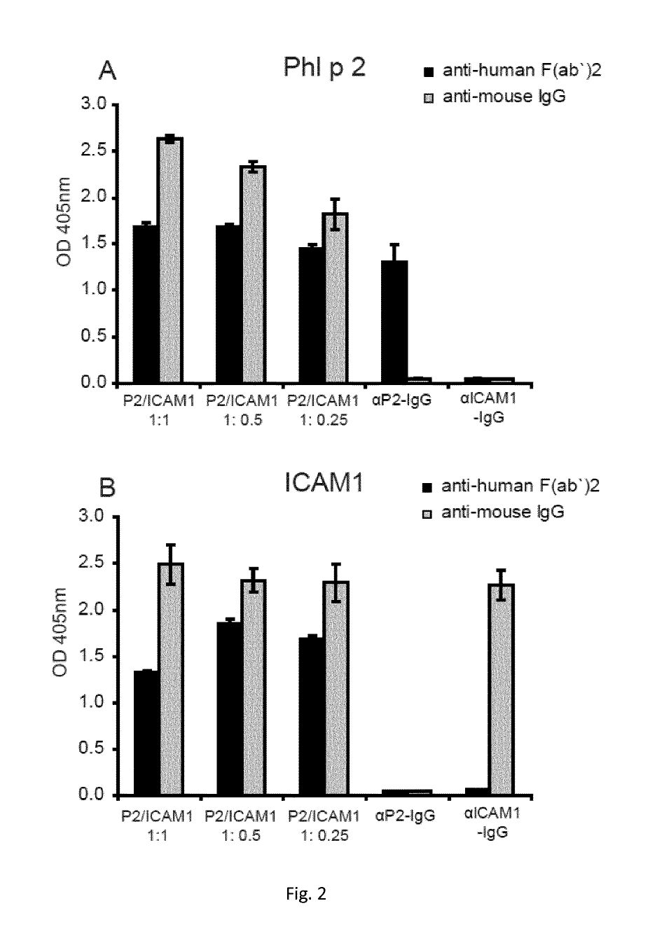

FIG. 2. Reactivity of P2/ICAM1 with Phl p 2 and ICAM1. Conjugates formed with different ratios of anti-Phl p 2 and anti-ICAM1 (x-axes: P2/ICAM1: 1:1; 1:0.5; 1:025), anti-Phl p 2 (αP2-IgG) or anti-ICAM1 (αICAM1-IgG) alone were tested for reactivity with Phl p 2 (A) or ICAM1 (B) and detected with anti-human F(ab′)₂ antibodies (black bars) or anti-mouse IgG (gray bars). OD values (y-axes) corresponding to bound P2/ICAM1 conjugates are shown as means of triplicates +/−SD.

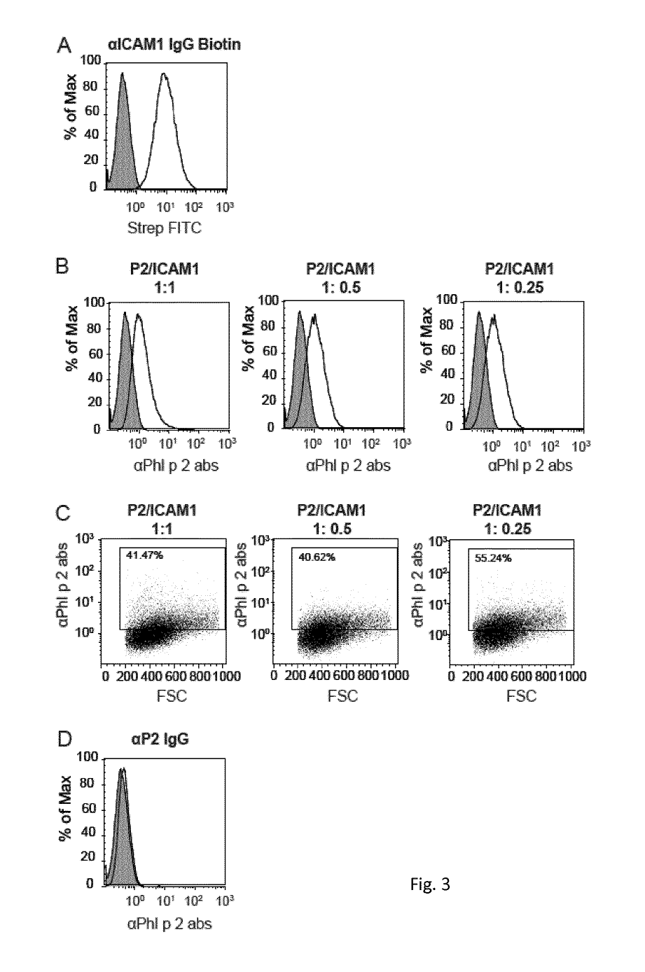

FIG. 3. Detection of ICAM1 or P2/ICAM1-captured Phl p 2 on respiratory epithelial cells using flow cytometry. (A) ICAM1 was detected on the surface of 16HBE14o- cells using ICAM1-specific antibodies (white graph) or an isotype control (gray graph). (B) Cells pre-incubated with different ratios of P2/ICAM1 (1:1; 1:0.5; 1:0.25) and Phl 1 p 2 were probed for cell-bound Phl p 2 with specific rabbit antibodies (white graphs) or the isotype control (gray graph). (C) Scatterplots showing the percentages of Phl p 2-reactive cells with three different ratios of P2/ICAM1. (D) Cells incubated with Phl p 2-specific human IgG₁ antibodies followed by Phl p 2 and Phl p 2-specific rabbit antibodies. Shown is one representative out of three independent experiments.

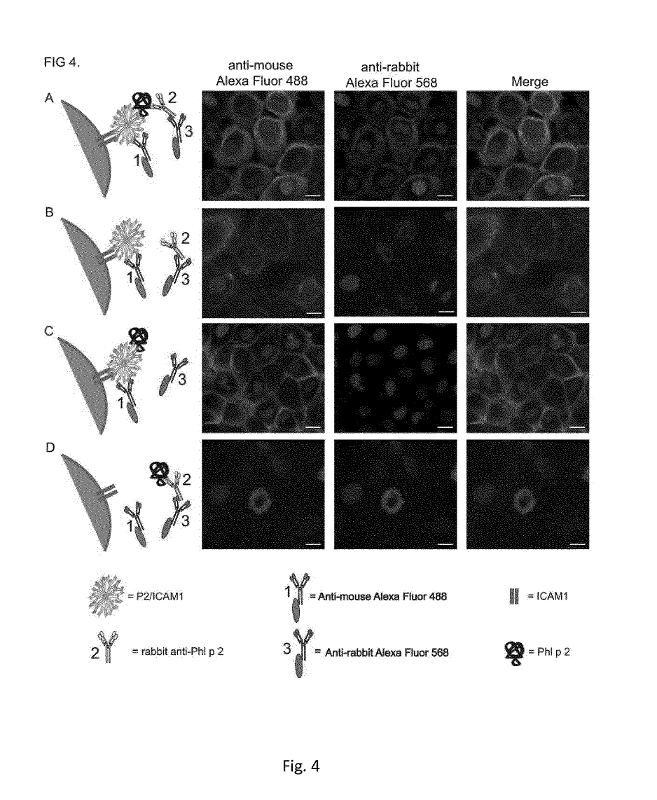

FIG. 4. Visualization of P2/ICAM1 and Phl p 2 on 16HBE14o- cells by immunofluorescence microscopy. Images showing cells incubated with different combinations of reactants (left margin and bottom), which were stained with Alexa Fluor®488-labelled anti-mouse antibodies and Alexa Fluor®568-labelled anti-rabbit antibodies to visualize αICAM1-IgG and Phl p 2, respectively. Nuclei were stained with DAPI and merged images are shown in the right column. Bars in the images represent 20 μm.

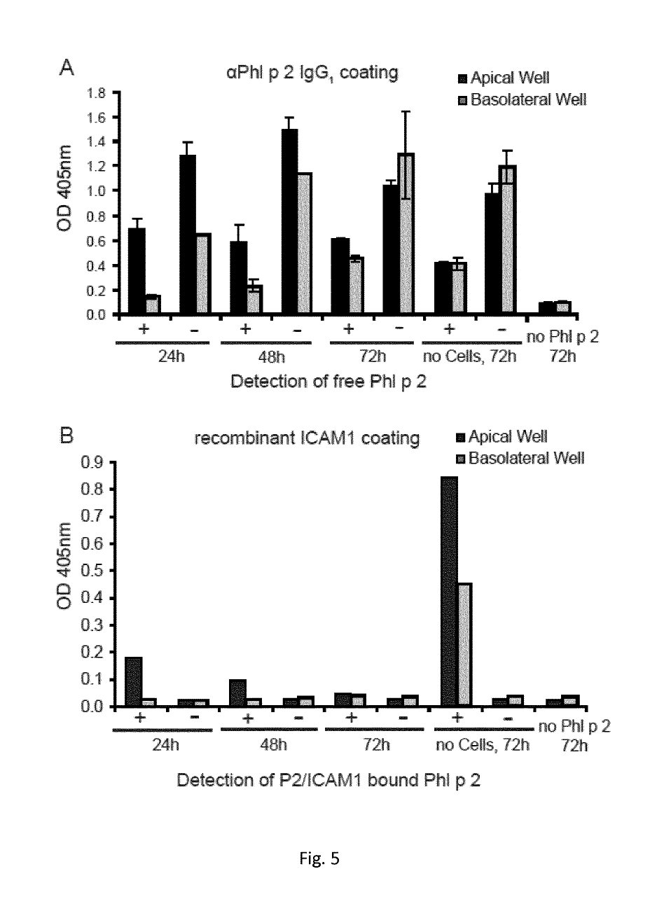

FIG. 5. Cell-bound P2/ICAM1 conjugates inhibit trans-epithelial migration of Phl p 2. (A) Detection of free Phl p 2 in apical (black bars) and basolateral (gray bars) compartments of cultured 16HBE14o- cell monolayers which had been pre-incubated with (+) or without (−) P2/ICAM1 conjugates. Concentrations of free Phl p 2 corresponding to OD values (y-axis) were measured at different points of time (x-axis: 24 h, 48 h, 72 h) in wells with or without cells (no cells). In the negative control, Phl p 2 was omitted (no Phl p 2). (B) Simultaneous detection of P2/ICAM1-Phl p 2 complexes in the very same samples as in (A). One out of three independent experiments giving comparable results is shown.

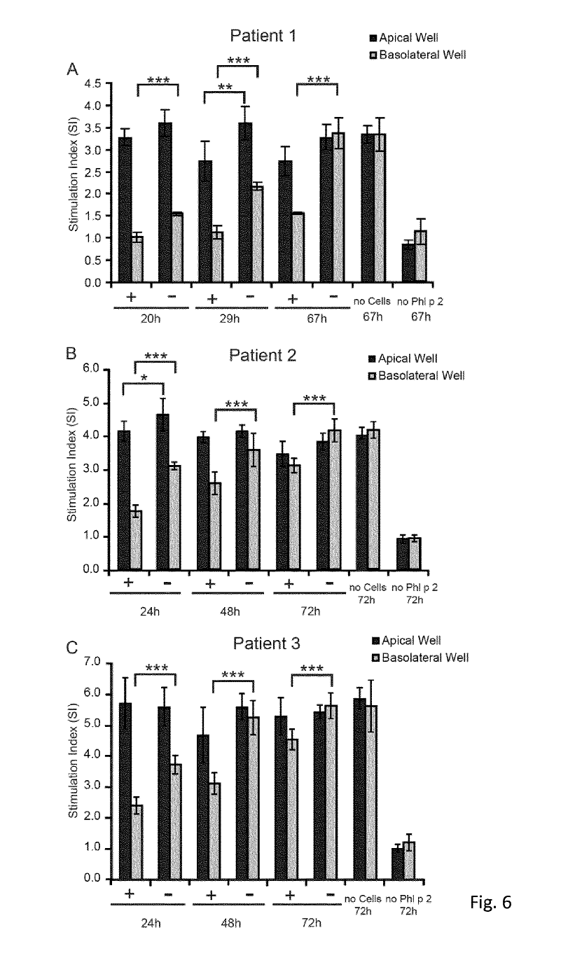

FIG. 6. Inhibition of trans-epithelial migration of Phl p 2 by P2/ICAM1 conjugates leads to decreased basophil activation with basolateral samples. Samples obtained from apical and basolateral compartments of cultures treated as in FIG. 5 were incubated with blood samples from three Phl p 2 allergic patients (A-C) and basophil activation was measured by determining up-regulation of the surface marker CD203c on basophils by flow cytometry. CD203c up-regulation expressed as stimulation index (SI) is displayed on the y-axis. Results are means of triplicates and error bars indicate standard deviation. *P<0.05, **<0.01, and *** P<0.001, ANOVA and linear contrasts.

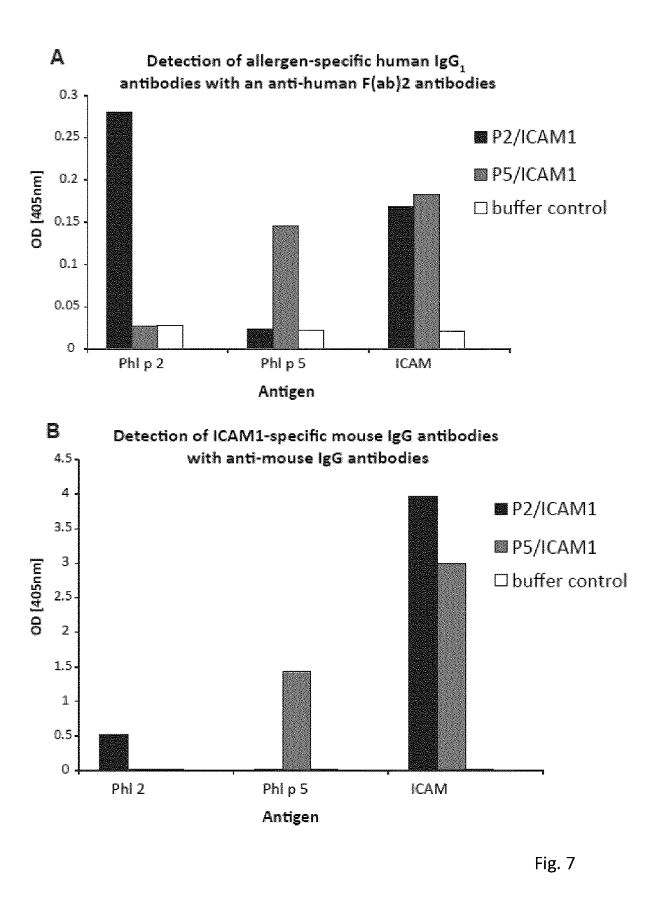

FIG. 7. Binding of bi-specific antibody conjugates P2/ICAM1 and P5/ICAM1 to immobilized antigens Phl p 2, Phl p 5 and recombinant ICAM1 in ELISAs. Bound P2/ICAM1, P5/ICAM1 were detected with anti-human F(ab)2 (A) or anti-mouse IgG (B) antibodies. Buffer containing 0.5% BSA was applied as negative control (buffer control).

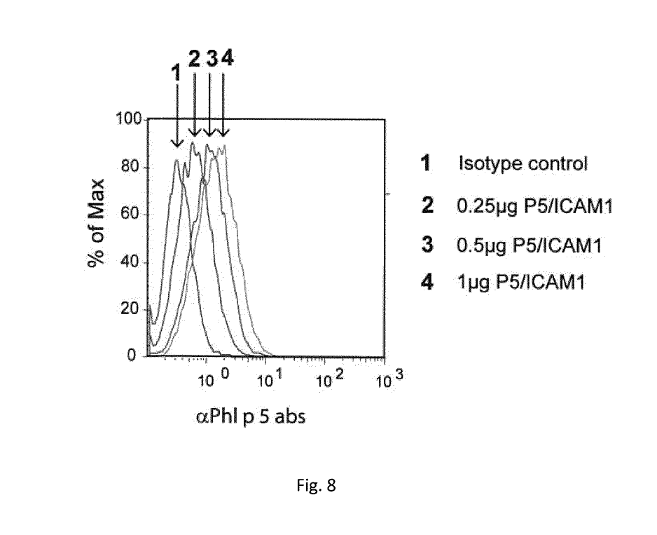

FIG. 8. Flow cytometry histograms of 16HBE14o- cells which were incubated with different concentrations of P5/ICAM1 (0.25 μg; 0.5 μg and 1 μg) followed by addition of a constant concentration of Phl p 5 (1 μg in 50 μl FACS buffer) and detection of bound Phl p 5 by rabbit anti-Phl p 5 and Alexa Fluor 488-labelled goat anti-rabbit antibodies. An anti-ICAM1 antibody isotype control was included.

### EXAMPLES

### Example 1: P2/ICAM1 and P5/ICAM1 Antibody Conjugates Specifically Bind Phl p 2 and ICAM1 and Phl p 5 and ICAM1, Respectively

Antibodies and Antibody Conjugation

A Phl p 2- and a Phl p 5-specific IgE Fab-fragment were isolated from an allergic patient as described in Steinberger et al. (J Biol Chem 271(1996):10967-10972). These Fabs were converted into fully human IgG₁ antibodies and stably expressed in CHO-K1 cells (see Flicker et al., J Immunol 165 (2000):3849-3859, and Flicker et al., Eur J Immunol. 32(2002):2156-62). These Phl p 2- and Phl p 5-specific IgG₁ were purified via Protein G affinity chromatography (Thermo Fisher Scientific, Pierce, USA). The Phl p 2- and Phl p 5-specific IgG₁ antibodies were conjugated with Lightning-Link® Streptavidin (Innova Biosciences, UK). A biotin-labeled ICAM1-specific mouse IgG₁ antibody was purchased from Abcam (UK). To form bi-specific antibody conjugates, termed P2/ICAM1 and P5/ICAM1, three different ratios (1:1, 1:0.5, 1:0.25) of the Phl p 2-specific human IgG₁ and a 1:1 ratio of Phl p 5-specific human IgG₁, respectively, and the ICAM1-specific mouse IgG₁ were co-incubated for at least one hour at room temperature.

ELISA Evaluation of the Binding Specificities of P2/ICAM1 Conjugates and P5/ICAM1 Conjugates

ELISA plates (Nunc Maxi-Sorp, Roskilde, Denmark) were coated with recombinant ICAM1 (R&D Systems, Minneapolis, USA) or recombinant Phl p 2 or Phl p 5 (Biomay AG, Austria) (5 μg/ml in 100 mM NaHCO₃, pH=9.6). Plates were incubated for 1 h at 37° C., washed twice with phosphate-buffered saline (PBS) containing 0.05% (v/v) Tween-20 (PBST) and saturated with PBST containing 3% (w/v) bovine serum albumin (BSA). P2/ICAM1 and P5/ICAM1 (1 μg/ml) were applied overnight at 4° C. Bound bi-specific antibody conjugates were detected either with horseradish peroxidase-conjugated rat anti-mouse IgG₁ antibodies (BD PharMingen, San Diego, Calif., USA) or with alkaline phosphatase-conjugated goat anti-human F(ab′)₂ antibodies (Pierce), both diluted 1:5000 in PBST containing 1% (w/v) BSA. Optical density (OD) measurements were carried out on ELISA reader Spectramax Plus (Molecular Devices, Sunnyvale, Calif., USA) at 405 nm. Results are means of triplicates. Error bars indicate standard deviations.

Results:

P2/ICAM1

Antibody conjugates, termed P2/ICAM1 consisting of a Phl p 2- (i.e., P2) and an ICAM1-specific monoclonal antibody (i.e., ICAM1) were formed through streptavidin-biotin coupling (i.e., conjugation) using different ratios of the individual components (P2/ICAM1: 1:1, 1:0.5, 1:0.25) (FIG. 1). In a first series of experiments it was investigated if formed antibody conjugates retain binding specificity for Phl p 2 and ICAM1 (FIG. 2). Conjugates consisting of different ratios of the monoclonal antibodies showed comparable binding to ELISA plate-bound Phl p 2 with the non-conjugated anti-Phl p 2 antibody when detected with anti-human F(ab′)₂ indicating that the latter was fully functional in the conjugate (FIG. 2 A). Phl p 2-bound conjugates could be also detected with the anti-mouse IgG antibody with decreasing intensities reflecting the proportion of the ICAM1-specific antibody in the conjugate (FIG. 2 A). No binding to Phl p 2 was detected with anti-mouse IgG antibodies when non-conjugated Phl p 2-specific antibodies were tested or when anti-ICAM1 antibodies were tested with coated Phl p 2 (FIG. 2 A). Binding of conjugate to ICAM1 could be demonstrated by detecting the Phl p 2-specific antibody as well as the ICAM1-specific antibody (FIG. 2 B). The intensities of binding with the conjugates were similar as for the non-conjugated ICAM1-specific antibody. No binding of the non-conjugated ICAM1-specific antibody to ICAM1 was found with anti-human F(ab′)₂. Also no reactivity of the non-conjugated Phl p 2-specific antibody to ICAM1 was observed with any of the detection systems (FIG. 2 B).

P5/ICAM1:

Antibody conjugates, termed P5/ICAM1 consisting of a Phl p 5- (i.e., P5) and an ICAM1-specific monoclonal antibody (i.e., ICAM1) were also formed through streptavidin-biotin coupling (i.e., conjugation) using a 1:1 ratio of the individual components. Additional experiments with the new formed conjugate (P5/ICAM1) were performed. In ELISA experiments it was demonstrated that the P5/ICAM1 conjugate binds specifically to Phl p 5 but not to Phl p 2 immobilized on ELISA plates (FIG. 7). It was further confirmed that the P2/ICAM1 conjugate that was used in all experiments specifically recognized Phl p 2 but not Phl p (FIG. 7). Additionally it was shown that P2/ICAM1 and P5/ICAM1 bind to recombinant ICAM1 (FIG. 7). The bi-specificity for both conjugates using either detection antibodies specific for allergen-specific human IgG1 (AP labelled anti-human F(ab)2) (FIG. 7A) or ICAM1-specific antibodies (HRP labelled anti-mouse antibodies) (FIG. 7B) was demonstrated.

### Example 2: Phl p 2 and Phl p 5 can be Immobilized Via P2/ICAM1 and P5/ICAM1, Respectively, onto Respiratory Epithelial Cells

Flow Cytometry

P2/ICAM1

The epithelial cell line 16HBE14o- was derived from human bronchial surface epithelial cells and cultivated. Aliquots of approximately 150.000 16HBE14o- cells were incubated with 1 μg of P2/ICAM1 in 50 μl FACS buffer (PBS supplemented with 2% w/v BSA) for 30 min on ice. After washing, aliquots of 1 μg Phl p 2 in 50 μl FACS buffer were added and cells were again incubated for 30 min on ice. After further washing, cells were incubated with Phl p 2-specific rabbit antibodies and an Alexa Fluor®488-labelled goat anti-rabbit antibody (Molecular Probes, Life Technologies, Eugene, Oreg., US). Biotin conjugated anti-ICAM1 antibody was visualized with Streptavidin conjugated to Alexa Fluor®488 (Molecular Probes). 7-AAD-dye (Beckman Coulter, Brea, Calif., US) was used for staining of dead cells. Controls included the biotin conjugated anti-ICAM1 antibody isotype control (mouse IgG₁ MOPC-21, Abcam) or the Phl p 2-specific antibody conjugated to Streptavidin followed by Phl p 2 and Phl p 2-specific rabbit antibodies. Cells were analysed on a FC500 Flow Cytometer (Beckman-Coulter) counting at least 30.000 cells per sample in duplicates and were evaluated with FlowJo version 7.2.5 (Treestar, Ashland, Oreg., US).

P5/ICAM1

Aliquots of approximately 150.000 16HBE14o- cells were incubated with 1 μg, 0.5 μg and 0.25 μg of P5/ICAM1 (1:1) in 50 μl FACS buffer (PBS supplemented with 2% w/v BSA) for 30 min on ice. After washing, aliquots of 1 μg Phl p 5 in 50 μl FACS buffer were added and cells were again incubated for 30 min on ice. After further washing, cells were incubated with Phl p 5-specific rabbit antibodies and an Alexa Fluor®488-labelled goat anti-rabbit antibody (Molecular Probes, Life Technologies, Eugene, Oreg., US). Control included the biotin conjugated anti-ICAM1 antibody isotype control (mouse IgG1 MOPC-21, Abcam). Cells were analysed on a FC500 Flow Cytometer (Beckman-Coulter) counting at least 30.000 cells per sample in duplicates and were evaluated with FlowJo version 7.2.5 (Treestar, Ashland, Oreg., US).

Immunofluorescence Microscopy

16HBE14o- cells, which had been cultured as described above were seeded on ibiTreat tissue culture-treated μ-dishes (Ibidi GmbH, Munich, Germany) and grown for 2 days to approximately 50% confluence. Cells were washed twice with PBS, cooled to 4° C. and further processed at 4° C. unless stated otherwise. Antibody-allergen complexes were formed by co-incubation of 5 μg P2/ICAM1 and 1 μg Phl p 2 in 300 μl PBS for 30 min at room temperature and subsequently incubated with the cells at 4° C. for 30 min. Unbound complexes were removed by washing with PBS and cells were then incubated with Phl p 2-specific rabbit antibodies diluted 1:1000 in PBS. Cells were washed with PBS, fixed with 4% formaldehyde solution for 20 min at room temperature and remaining aldehyde groups were quenched with 50 mM ammonium chloride in PBS for 10 min. Unspecific binding sites were blocked with 10% goat serum in PBS overnight at 4° C. To detect cell-bound P2/ICAM1, Alexa Fluor® 488 goat anti-mouse IgG (Molecular Probes) diluted 1:1000 in PBS containing 10% goat serum was added to the cells for 1 hour at room temperature. Phl p 2 bound to P2/ICAM1 was visualized with Alexa Fluor® 568 goat anti-rabbit IgG (Molecular Probes) applied at a dilution of 1:1000 in PBS for 1 hour at room temperature. Finally, nuclei were stained with 1 ug/ml DAPI (Molecular Probes) in PBS. Cells were washed twice with PBS between individual incubations. Staining with fluorescent antibodies alone was done to assess background signals. Controls omitting either Phl p 2, Phl p 2-specific rabbit antibodies or P2/ICAM1 were included. Wide-field fluorescence imaging of fixed cells was carried out using a Zeiss Axio Observer Z1 inverted fluorescence microscope equipped with an oil immersion 40×lens (Pan Apochromat, 1.4 NA, Carl Zeiss Inc., Oberkochen, Germany) and the Zeiss AxioVision Software package (Release 4.8).

Results

P2/ICAM1

To study if P2/ICAM1 can be used to immobilize Phl p 2 on the surface of respiratory epithelial cells, 16HBE14o- cells, which express ICAM1 on their surface as shown by FACS analysis (FIG. 3 A), were used as surrogate. Cells were first incubated with P2/ICAM1 conjugates (different ratios of P2/ICAM1: 1:1, 1:0.5, 1:0.25) and, after washing, exposed to Phl p 2. Using Phl p 2-specific rabbit antibodies immobilization of the Phl p 2 allergen onto the cell surface via P2/ICAM1 could be demonstrated (FIG. 3 B). A scatter plot analysis showed that immobilization was best (i.e., more than 50% of the cells had Phl p 2 bound to their cell surface) when the ratio of Phl p 2-specific antibody to ICAM1-specific antibody was 1:0.25 (FIG. 3 C). No staining of cells was observed when the non-conjugated Phl p 2-specific antibody followed by Phl p 2 was added to cells and exposed to Phl p 2-specific rabbit antibodies (FIG. 3 D).

To study the immobilization of Phl p 2 via P2/ICAM1 on the cells in more depth, immunofluorescence microscopy experiments were conducted. Images showed co-localization (FIG. 4 A, merge) of Phl p 2 (Alexa Fluor®568) and P2/ICAM1 (Alexa Fluor®488) on the cell surface. When either Phl p 2 (FIG. 4 B) or Phl p 2-specific rabbit antibodies (FIG. 4 C) were omitted, no binding of Alexa Fluor® 568 goat anti-rabbit IgG was observed. Whenever P2/ICAM1 was added to the cells it was detected with Alexa Fluor 488 goat anti-mouse IgG specific for αICAM 1 IgG (FIG. 4 A, B, C). When P2/ICAM1 was omitted, detection antibodies did not bind (FIG. 4 D). No staining of cells was found when only secondary antibodies alone were applied.

P5/ICAM1

The newly formed P5/ICAM1 conjugates was used in additional FACS experiments similar to those shown in Figure E2 of Madritsch C et al. (J Allergy Clin Immunol 136(2015):490-493) to confirm that another bi-specific anti-ICAM1/anti-allergen conjugate is able to immobilize the allergen on the surface of respiratory epithelial cells. Cells were first incubated with P5/ICAM1 conjugates (1 μg; 0.5 μg and 0.25 μg) and, after washing, exposed to Phl p 5. Using Phlp 5-specific rabbit antibodies immobilization of the Phl p 5 allergen onto the cell surface via P5/ICAM1 could be demonstrated (FIG. 8).

### Example 3: P2/ICAM1 Conjugates Prevent Migration of Phl p 2 Through Respiratory Epithelial Cell Layers

ELISA Differentiating Free Phl p 2 from P2/ICAM1-Bound Phl p 2 in Culture Samples

To measure free Phl p 2 in culture samples the Phl p 2-specific human IgG₁ was coated (1 μg/ml in 100 mM NaHCO₃, pH=9.6) to ELISA plates, which were washed and blocked as described above. Phl p 2 was then detected with rabbit anti-Phl p 2 antibodies (1:1000). Bound rabbit antibodies were visualized with a horseradish peroxidase-conjugated donkey anti-rabbit antibody diluted 1:2000 (GE Healthcare, Little Chalfont, UK).

To detect P2/ICAM1-bound Phl p 2, recombinant ICAM1 (1 μg/ml in 100 mM NaHCO₃, pH=9.6) was coated to ELISA plates, which were washed and blocked as describe above. Cell culture supernatants were applied and P2/ICAM1-bound Phl p 2 was detected with rabbit anti-Phl p 2 antibodies as describe above. Results represent means of duplicates with error bars indicating standard deviation.

Cell Culture and Transwell Experiments

The epithelial cell line 16HBE14o- was derived from human bronchial surface epithelial cells and cultivated. TER baseline ranged consistently from 110 to 120Ω/cm² for transwells filled with medium without cells and was measured with an ohm voltmeter (Millipore, USA) When cells reached a TER value of 400Ω/cm² (baseline subtracted) IFN-γ (50 ng/ml, Pepro Tech Inc; USA) was added in all cultures to allow detectable allergen migration. Aliquots of 5 μg P2/ICAM1 (ratio 1:0.25) were applied to the apical chambers for three hours at 37° C. Supernatants were removed and 10 ng Phl p 2 in 0.5 ml medium was added to the apical compartment for 20 h-72 h at 37° C. The effect of P2/ICAM1 on Phl p 2 apical-to-basolateral trans-epithelial migration was assessed by comparing P2/ICAM1 treated wells with untreated control wells. In addition, the transmigration of Phl p 2 as well as of P2/ICAM1-bound Phl p 2 through transwell membranes without cells was assessed. Samples from apical and basolateral compartments were tested for the presence of free Phl p 2 and/or P2/ICAM1-bound Phl p 2 by ELISA.

Results:

In a next series of experiments it was investigated whether P2/ICAM1 can prevent the apical-to-basolateral penetration of Phl p 2 through a layer of cultured respiratory epithelial cells. For this purpose, the apical compartments of cells were pre-incubated with or without P2/ICAM1 conjugates. Subsequently the Phl p 2 allergen was added to the apical side and its penetration through the layer into the basolateral compartment was measured with a catching ELISA differentiating free Phl p 2 from P2/ICAM1-bound Phl p 2 (FIG. 5 A, B). The apical addition of P2/ICAM1 strongly prevented the penetration of Phl p 2 over the full period of analysis (i.e., for 72 hours) into the basolateral compartment (see FIG. 5 A, gray bars +) when compared to conditions without addition of P2/ICAM1 (FIG. 5 A, gray bars −). When Phl p 2 and P2/ICAM1 conjugates were omitted, no Phl p 2 signal was detected (FIG. 5 A, no Phl p 2, 72 h).

In this experimental set up the apical-to-basolateral penetration of the complex between P2/ICAM1 and the Phl p 2 allergen was also studied (FIG. 5 B). This was achieved with a sandwich ELISA using recombinant ICAM1 for catching the P2/ICAM1 conjugates and detecting bound Phl p 2 with rabbit anti-Phl p 2 antibodies. In fact, when cell monolayers were used no relevant penetration of P2/ICAM1-Phl p 2 complexes into the basolateral wells was found (FIG. 5 B). Therefore, we also performed experiments without cells where P2/ICAM1 and Phl p 2 was added simultaneously showing that P2/ICAM1-Phl p 2 immune complexes were formed and penetrated into the basolateral well when no cells were present (FIG. 5 B, no cells, 72 h).

### Example 4: Prevention of Trans-Epithelial Allergen Migration by P2/ICAM1 Leads to Decreased Basophil Activation

Basophil Activation Assays

Basophil activation was assessed in vitro by measuring up-regulation of CD203c expression. Heparinized blood from grass pollen allergic patients with IgE antibodies against Phl p 2 were obtained after informed consent was given. Blood aliquots (90 μl) were incubated with either samples from the transwell experiments or for control purposes with a monoclonal anti-IgE antibody (1 mg/mL; Immunotech, Vaudreuil-Dorion, Quebec, Canada) or PBS for 15 minutes at 37° C. Allergen-induced up-regulation of CD203c was calculated from mean fluorescence intensities (MFIs) obtained with stimulated (MFIstim) and unstimulated (MFIcontrol) cells and expressed as stimulation index (SI) (MFIstim/MFIcontrol). Cells were analyzed by 2-color flow cytometry on a FACScan (BD Biosciences, USA).

Results

To test whether a reduction of trans-epithelial allergen migration by P2/ICAM1 has an effect on basophil activation basophil activation experiments using samples from the apical and basolateral compartments taken from three independent transwell experiments, were performed. Results from these experiments with samples of three representative allergic patients are shown in FIG. 6 A, B, C. Comparing the extent of basophil activation induced with basolateral samples showed a significant and consistent reduction of basophil activation for samples taken from cultures where P2/ICAM1 had been added to the apical compartments (FIG. 6 A, B, C, gray bars +) compared to those where P2/ICAM1 conjugates had been omitted (gray bars −). This effect was observed at each of the analyzed time points. The extent of basophil-activation by samples taken from the apical compartments (black bars) was comparable and almost identical to that obtained with basolateral samples from cell-free preparations (FIG. 6 A, B, C, no cells). Basophil activation was not observed when Phl p 2 was absent from the cultures (FIG. 6 A, B, C, no Phl p 2).

1. An antibody construct comprising at least one antigen binding site specific for ICAM1 and at least one antigen binding site specific for at least one allergen.

2. The antibody construct of claim 1, wherein the antibody construct is a complex, conjugate or fusion protein comprising an antigen binding site specific for ICAM1 and an antigen binding site specific for an allergen.

3. The antibody construct of claim 1 comprising an antigen binding site specific for ICAM1 chemically cross-linked to an antigen binding site specific for an allergen.

4. The antibody construct of claim 1, wherein the antibody construct is a bispecific antibody, a bispecific diabody or a bispecific (Fab)₂ fragment.

5. The antibody construct of claim 1, wherein the allergen is an inhalation or food allergen.

6. The antibody construct of claim 5, wherein the inhalation allergen is selected from the group consisting of pollen allergens, mold allergens, mite allergens and animal allergens, preferably pet allergens.

7. The antibody construct of claim 1, wherein the allergen is selected from the group consisting of a pollen allergen, mite allergen, cat allergen, dog allergen, insect bite/sting allergen, legume allergen, fruit allergen, nut allergen, milk allergen and fish allergen.

8. The antibody construct of claim 1, wherein the allergen is a pollen allergen selected from the group of a grass pollen allergen, preferably the grass pollen allergen Phl p 1, Phl p 2, Phl p 4, Phl p 5, Phl p 6, Phl p 7, Phl p 11, Phl p 12 or Phl p 13, a weed allergen, preferably the ragweed allergen Amb a 1, Amb a 2, Amb a 3, Amb a 4, Amb a 5, Amb a 6, Amb a 7, Amb a 8, Amb a 9 or Amb a 10, a mugwort allergen, preferably Art v 1, Art v 2, Art v 3, Art v 4, Art v 5 or Art v 6, a Paritaria pollen allergen, preferably Par j 1, Par j 2, Par j 3 or Par j 4, a olive pollen allergen, preferably Ole e 1, Ole e 2, Ole e 3, Ole e 4, Ole e 5, Ole e 6, Ole e 7, Ole e 8, Ole e 9, Ole e 10 or Ole e 11, a pollen allergen from Saltwort thistle, preferably Sal k 1, Sal k 2, Sal k 3, Sal k 4 or Sal k 5, and a tree pollen, preferably a Cypress pollen, preferably Cha o 1 or Cha o 2, or a cedar pollen, preferably Jun a 1, Jun a 2 or Jun a 3, or a birch pollen allergen, preferably Bet v 1.

9. The antibody construct of claim 1, wherein the allergen is Phl p 2 or Phl p 5.

10. The antibody construct of claim 1, wherein the allergen is a mite allergen, preferably Der p 1, Der p 2, Der p 3, Der p 4, Der p 5, Der p 6, Der p 7, Der p 8, Der p 9, Der p 10, Der p 11, Der p 14, Der p 15, Der p 18, Der p 20, Der p 21 and Der p 23.

11. The antibody construct of claim 1, wherein the allergen is an animal, preferably pet allergen, selected from a group consisting of a cat allergen, preferably Fel d 1, Fel d 2, Fel d 3, Fel d 4, Fel d 5, Fel d 6, Fel d 7 and Fel d 8, a dog allergen, preferably Can f 1, Can f 2, Can f 3, Can f 4 and Can f 5, and a cockroach allergen, preferably Bla g 1, Bla g 2, Bla g 3, Bla g 4, Bla g 5, Blag 6, Blag 7, Bla g 8, Bla g 11, Per a 1, Per a 2, Per a 3, Per a 4, Per a 5, Per a 6, Per a 7, Per a 9 and Per a 10.

12. The antibody construct of claim 1, wherein the antibody construct comprises at least one immunoglobulin or at least one immunoglobulin fragment comprising an antigen binding site for ICAM1 and at least one immunoglobulin or at least one immunoglobulin fragment comprising an antigen binding site for at least one allergen.

13. A pharmaceutical composition comprising the antibody construct of claim 1.

14. The pharmaceutical composition of claim 13, wherein the composition is adapted for intranasal, oral or ocular administration or for inhalation.

15. The pharmaceutical composition of claim 13 for the use of treating and/or preventing an allergy.

16. The pharmaceutical composition of claim 13 for the use of preventing, reducing and/or alleviating allergic symptoms in a patient.

17. The pharmaceutical composition for the use of claim 15, wherein the pharmaceutical composition is administered by inhalation.


SEQUENCE LISTING (94 sequences)

SEQ ID NO: 1 - protein, 114 aa, Artificial Sequence
  Antigen binding site or immunoglobulin or immunoglobulin fragment specific for Phl p 2
LESGPGLVKPAQTLSLSCAVSGGSIRSGGYYWSWIRQHPGKGLEWIGYIYHSGNTYYNPSLKSRIAMSVDTSENKFSLRLNSVTAADTAVYYCARLDGYTLDIWGQGTLVTVSS

SEQ ID NO: 2 - protein, 106 aa, Artificial Sequence
  Antigen binding site or immunoglobulin or immunoglobulin fragment specific for Phl p 2
ELTQSPSSLSASVGDRVTISCRASQRINTYLNWYQHKPGKAPKLLIYAASSLQSGVPSRFSGSGYGTDFTLTISSLQPEDFASYYCQESLSASYTFGQGTKVEIKR

SEQ ID NO: 3 - protein, 259 aa, Artificial Sequence
  Antigen binding site or immunoglobulin or immunoglobulin fragment specific for Phl p 2
MAQVQLQESGPGLVKPAQTLSLSCAVSGGSIRSGGYYWSWIRQHPGKGLEWIGYIYHSGNTYYNPSLKSRIAMSVDTSENKFSLRLNSVTAADTAVYYCARLDGYTLDIWGQGTLVTVSSGGGGSGGGGSGGGGSETTLTQSPSSLSASVGDRVTISCRASQRINTYLNWYQHKPGKAPKLLIYAASSLQSGVPSRFSGSGYGTDFTLTISSLQPEDFASYYCQESLSASYTFGQGTKVEIKRAAAGAPVPYPDPLEPR

SEQ ID NO: 4 - protein, 114 aa, Artificial Sequence
  Antigen binding site or immunoglobulin or immunoglobulin fragment specific for Phl p 2
LESGPGLVKPSQTLSLTCTVSGGSIRSGGYYWSWVRQPPGKGLEWIGNIYHSGNTYYNPSLKSRITMSVDTSKNHFSLRLTSVTAADTAVYYCARSDGYTLDNWGQGTLVTVSS

SEQ ID NO: 5 - protein, 106 aa, Artificial Sequence
  Antigen binding site or immunoglobulin or immunoglobulin fragment specific for Phl p 2
ELTQSPSSLSASVGDRVTITCRARQSISTYLNWYQQKPGKAPKLLIWSASNLQSGVPSRFSGSGSGTEFTLTISNLQPEDFASYYCQQSYTTLYTFGPGTKLEIKR

SEQ ID NO: 6 - protein, 114 aa, Artificial Sequence
  Antigen binding site or immunoglobulin or immunoglobulin fragment specific for Phl p 2
LESGPGLVKPSQTLSLTCTVSGGSIRSGGYYWSWIRQPPGKGLEWIGYIYHSGNTYYNPSLKSRVTMSVDTSKNHFSLRLSSVTAADTAVYYCARSDGYTLDNWGQGTLVTVSS

SEQ ID NO: 7 - protein, 106 aa, Artificial Sequence
  Antigen binding site or immunoglobulin or immunoglobulin fragment specific for Phl p 2
ELTQSPSSVSASVGDRVTITCRASQGISSWLAWYQQKPGKAPKLLIYSASSLQSGVPSRFSGSGSGTDFSLTISSLQPEDSATYYCQQANSFPYTFGQGTKVEIKR

SEQ ID NO: 8 - protein, 10 aa, Artificial Sequence
  Antigen binding site or immunoglobulin or immunoglobulin fragment specific for Phl p 2
GGSIRSGGYY

SEQ ID NO: 9 - protein, 7 aa, Artificial Sequence
  Antigen binding site or immunoglobulin or immunoglobulin fragment specific for Phl p 2
IYHSGNT

SEQ ID NO: 10 - protein, 11 aa, Artificial Sequence
  Antigen binding site or immunoglobulin or immunoglobulin fragment specific for Phl p 2
ARLDGYTLDIW

SEQ ID NO: 11 - protein, 6 aa, Artificial Sequence
  Antigen binding site or immunoglobulin or immunoglobulin fragment specific for Phl p 2
QRINTY

SEQ ID NO: 12 - protein, 3 aa, Artificial Sequence
  Antigen binding site or immunoglobulin or immunoglobulin fragment specific for Phl p 2
AAS

SEQ ID NO: 13 - protein, 9 aa, Artificial Sequence
  Antigen binding site or immunoglobulin or immunoglobulin fragment specific for Phl p 2
QESLSASYT

SEQ ID NO: 14 - protein, 10 aa, Artificial Sequence
  Antigen binding site or immunoglobulin or immunoglobulin fragment specific for Phl p 2
ARSDGYTLDN

SEQ ID NO: 15 - protein, 6 aa, Artificial Sequence
  Antigen binding site or immunoglobulin or immunoglobulin fragment specific for Phl p 2
QSISTY

SEQ ID NO: 16 - protein, 3 aa, Artificial Sequence
  Antigen binding site or immunoglobulin or immunoglobulin fragment specific for Phl p 2
SAS

SEQ ID NO: 17 - protein, 9 aa, Artificial Sequence
  Antigen binding site or immunoglobulin or immunoglobulin fragment specific for Phl p 2
QQSYTTLYT

SEQ ID NO: 18 - protein, 6 aa, Artificial Sequence
  Antigen binding site or immunoglobulin or immunoglobulin fragment specific for Phl p 2
QGISSY

SEQ ID NO: 19 - protein, 9 aa, Artificial Sequence
  Antigen binding site or immunoglobulin or immunoglobulin fragment specific for Phl p 2
QQANSFPYT

SEQ ID NO: 20 - protein, 117 aa, Artificial Sequence
  Antigen binding site or immunoglobulin or immunoglobulin fragment specific for Phl p 1
LESGGGWVQPGRSLRLSCAASGFTFDDHAMHWVRQAPGKGLEWVAGVSWNSGTRAYADSVKGRFTISRDNAKNSLYLEMKSLRAEDTAIYYCARDMRAATPYSFDYWGQGVLVTVSS

SEQ ID NO: 21 - protein, 108 aa, Artificial Sequence
  Antigen binding site or immunoglobulin or immunoglobulin fragment specific for Phl p 1
ELVMTQSPSSLSASVGDRVTITCRASQTFNNYLNWYQQKPGKAPNLLIYAASTLRRGIPSRFSGSGSGTYFTLTINGLQPEDFATYYCQQSYSTPLTFGGGTKVDIKR

SEQ ID NO: 22 - protein, 117 aa, Artificial Sequence
  Antigen binding site or immunoglobulin or immunoglobulin fragment specific for Phl p 1
LESGGGLVQPGGSLRLSCAASGFTFDDHAMHWVRQTPEKGLQWVSGISWNSGVVAYADSVKGRFSISRDNAKNSLYLQMNNLIPEDTAVYYCVRDFRAASPYFFDYWGQGTRVTVSS

SEQ ID NO: 23 - protein, 106 aa, Artificial Sequence
  Antigen binding site or immunoglobulin or immunoglobulin fragment specific for Phl p 1
ELTQSPSSLSASVGDRVTITCRASQSIGNYLNWYQQKPGKAPNLLIYAASSLQSGVPSRFSGSGSGTDFTLTISSLQREDFATYYCQQSNRTPITFGQGTRLEIKG

SEQ ID NO: 24 - protein, 262 aa, Artificial Sequence
  Antigen binding site or immunoglobulin or immunoglobulin fragment specific for Phl p 1
MAQVNLRESGGGLVQPGGSLRLSCAASGFTFDDHAMHWVRQTPEKGLQWVSGISWNSGVVAYADSVKGRFSISRDNAKNSLYLQMNNLIPEDTAVYYCVRDFRAATPYFFDYWGQGTLVTVSSGGGGSGGGGSGGGGSETTLTQSPSSLSASVGDRVTITCRASQSIGNYLNWYQQKPGKAPNLLIYAASSLQSGVPSRFSGSGSGTDFTLTISSLQREDFATYYCQQSNRTPITFGQGTRLEIKRAAAGAPVPYPDPLEPR

SEQ ID NO: 25 - protein, 117 aa, Artificial Sequence
  Antigen binding site or immunoglobulin or immunoglobulin fragment specific for Phl p 1
LESGGGWVQPGRSLRLSCSASGLTFDDYAMHWVRQAPGKGLEWVSGISWNSGVRAYADSVKGRFTISRDNGKNSLYLQMNSLRPEDTALYYCAKDIRAATPYALDHWGQGVLVTVSS

SEQ ID NO: 26 - protein, 108 aa, Artificial Sequence
  Antigen binding site or immunoglobulin or immunoglobulin fragment specific for Phl p 1
ELVMTQSPSSLSASEGDRVTITCRASRTIYNYLNWYQQKPGRAPKLLIHAASTLQDGVPSRFSGSGSGTEFTLTISGLQSEDFATYYCQQSHGTPLTFGGGTTVDVKR

SEQ ID NO: 27 - protein, 259 aa, Artificial Sequence
  Antigen binding site or immunoglobulin or immunoglobulin fragment specific for Phl p 1
MAQVQLQESGGGLVQPGGSLKLSCAASEFTFNNYWMSWVRQAPGKGLEWVANIKPDGSVKYYVDSVKGRFTISRDNAKNSLFLQMNSLRAEDTAVYFCARERQSGAFGLWGQGTTVTVSSGAGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRAAAGAPVPYPDPLEPR

SEQ ID NO: 28 - protein, 269 aa, Artificial Sequence
  Antigen binding site or immunoglobulin or immunoglobulin fragment specific for Phl p 1
MAQVNLRESGGGLVRPGRSLRLSCAASGFTFDDYAMHWVRQAPGKGLEWVSGISWNSGSIGYADSVKGRFTISRDNAKNSLYLQMNSLRAEDTAVYYCAREWDGTAYYGYTVGYWGQGTLVTVSSGGGGSGGGGSGGGGSDVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPPTFGQGTRLEIKRAAAGAPVPYPDPLEPR

SEQ ID NO: 29 - protein, 271 aa, Artificial Sequence
  Antigen binding site or immunoglobulin or immunoglobulin fragment specific for Phl p 1
MAQVQLQESGGGVVQPGRSLRLSCAASGFTFSSYAMSWVRQAPGKGLEWVSGISGSGGSTYYADSVKGRFTISRDNSKNTLYLQMNSLRAEDTAIYYCARVLRNQAAAGTIRWFDPWGQGTLVTVSSGGGGSGGGGSGGGGSDVVMTQSPLSLPVTLGQPASISCRSSQSLVYSDGNTYLNWFQQRPGQSPRRLIYKVSNRDSGVPDRFSGSGSGTDFTLKIRRVEAEDVGVYYCMQGTHWPPTFGQGTKLEIKRAAAGAPVPYPDPLEPR

SEQ ID NO: 30 - protein, 8 aa, Artificial Sequence
  Antigen binding site or immunoglobulin or immunoglobulin fragment specific for Phl p 1
GFTFDDHA

SEQ ID NO: 31 - protein, 8 aa, Artificial Sequence
  Antigen binding site or immunoglobulin or immunoglobulin fragment specific for Phl p 1
VSWNSGTR

SEQ ID NO: 32 - protein, 14 aa, Artificial Sequence
  Antigen binding site or immunoglobulin or immunoglobulin fragment specific for Phl p 1
ARDMRAATPYSFDY

SEQ ID NO: 33 - protein, 6 aa, Artificial Sequence
  Antigen binding site or immunoglobulin or immunoglobulin fragment specific for Phl p 1
QTFNNY

SEQ ID NO: 34 - protein, 9 aa, Artificial Sequence
  Antigen binding site or immunoglobulin or immunoglobulin fragment specific for Phl p 1
QQSYSTPLT

SEQ ID NO: 35 - protein, 8 aa, Artificial Sequence
  Antigen binding site or immunoglobulin or immunoglobulin fragment specific for Phl p 1
ISWNSGVV

SEQ ID NO: 36 - protein, 14 aa, Artificial Sequence
  Antigen binding site or immunoglobulin or immunoglobulin fragment specific for Phl p 1
VRDFRAASPYFFDY

SEQ ID NO: 37 - protein, 6 aa, Artificial Sequence
  Antigen binding site or immunoglobulin or immunoglobulin fragment specific for Phl p 1
QSIGNY

SEQ ID NO: 38 - protein, 9 aa, Artificial Sequence
  Antigen binding site or immunoglobulin or immunoglobulin fragment specific for Phl p 1
QQSNRTPIT

SEQ ID NO: 39 - protein, 8 aa, Artificial Sequence
  Antigen binding site or immunoglobulin or immunoglobulin fragment specific for Phl p 1
GLTFDDYA

SEQ ID NO: 40 - protein, 8 aa, Artificial Sequence
  Antigen binding site or immunoglobulin or immunoglobulin fragment specific for Phl p 1
ISWNSGVR

SEQ ID NO: 41 - protein, 14 aa, Artificial Sequence
  Antigen binding site or immunoglobulin or immunoglobulin fragment specific for Phl p 1
AKDIRAATPYALDH

SEQ ID NO: 42 - protein, 6 aa, Artificial Sequence
  Antigen binding site or immunoglobulin or immunoglobulin fragment specific for Phl p 1
RTIYNY

SEQ ID NO: 43 - protein, 9 aa, Artificial Sequence
  Antigen binding site or immunoglobulin or immunoglobulin fragment specific for Phl p 1
QQSHGTPLT

SEQ ID NO: 44 - protein, 8 aa, Artificial Sequence
  Antigen binding site or immunoglobulin or immunoglobulin fragment specific for Phl p 1
EFTFNNYW

SEQ ID NO: 45 - protein, 8 aa, Artificial Sequence
  Antigen binding site or immunoglobulin or immunoglobulin fragment specific for Phl p 1
IKPDGSVK

SEQ ID NO: 46 - protein, 11 aa, Artificial Sequence
  Antigen binding site or immunoglobulin or immunoglobulin fragment specific for Phl p 1
ARERQSGAFGL

SEQ ID NO: 47 - protein, 6 aa, Artificial Sequence
  Antigen binding site or immunoglobulin or immunoglobulin fragment specific for Phl p 1
QSISSY

SEQ ID NO: 48 - protein, 8 aa, Artificial Sequence
  Antigen binding site or immunoglobulin or immunoglobulin fragment specific for Phl p 1
GFTFDDYA

SEQ ID NO: 49 - protein, 8 aa, Artificial Sequence
  Antigen binding site or immunoglobulin or immunoglobulin fragment specific for Phl p 1
ISWNAGSI

SEQ ID NO: 50 - protein, 16 aa, Artificial Sequence
  Antigen binding site or immunoglobulin or immunoglobulin fragment specific for Phl p 1
AREWDGTAYYGYTVGY

SEQ ID NO: 51 - protein, 11 aa, Artificial Sequence
  Antigen binding site or immunoglobulin or immunoglobulin fragment specific for Phl p 1
QSLVYSDGNTY

SEQ ID NO: 52 - protein, 3 aa, Artificial Sequence
  Antigen binding site or immunoglobulin or immunoglobulin fragment specific for Phl p 1
KVS

SEQ ID NO: 53 - protein, 9 aa, Artificial Sequence
  Antigen binding site or immunoglobulin or immunoglobulin fragment specific for Phl p 1
MQGTHWPPT

SEQ ID NO: 54 - protein, 8 aa, Artificial Sequence
  Antigen binding site or immunoglobulin or immunoglobulin fragment specific for Phl p 1
GFTFSSYA

SEQ ID NO: 55 - protein, 8 aa, Artificial Sequence
  Antigen binding site or immunoglobulin or immunoglobulin fragment specific for Phl p 1
ISGSGGST

SEQ ID NO: 56 - protein, 18 aa, Artificial Sequence
  Antigen binding site or immunoglobulin or immunoglobulin fragment specific for Phl p 1
ARVLRNQAAAGTIRWEDP

SEQ ID NO: 57 - protein, 10 aa, Artificial Sequence
  Antigen binding site or immunoglobulin or immunoglobulin fragment specific for Phl p 1
QSLVYSDGNT

SEQ ID NO: 58 - protein, 276 aa, Artificial Sequence
  Antigen binding site or immunoglobulin or immunoglobulin fragment specific for Bet v 1
MAQVQLVQSGAEVKKPGSSVKVSCKASGGTFSSYAISWVRQAPGQGLEWMGRIIPILGIVNYAQKFQGRVTITADKSTSTAYMELSSLRSEDTAVYYCAREPLGYCSGGSCYPLHNGMDVWGQGTTVTVSSSGGGSGGGGSGGGGSDIQMTQSPDSLAVSLGERATINCKSSQSVLYSSNNKNYLAWYQQKPGQPPKLLFYWASTRESGVPDRFSGSGSGTDFTLTISSLQAEDVAVYYCQQYYTTPLTFGQGTKLEIKRAAAGAPVPYPDPLEPR

SEQ ID NO: 59 - protein, 8 aa, Artificial Sequence
  Antigen binding site or immunoglobulin or immunoglobulin fragment specific for Bet v 1
GGTFSSYA

SEQ ID NO: 60 - protein, 8 aa, Artificial Sequence
  Antigen binding site or immunoglobulin or immunoglobulin fragment specific for Bet v 1
IIPILGIV

SEQ ID NO: 61 - protein, 22 aa, Artificial Sequence
  Antigen binding site or immunoglobulin or immunoglobulin fragment specific for Bet v 1
AREPLGYCSGGSCYPLHNGMDV

SEQ ID NO: 62 - protein, 12 aa, Artificial Sequence
  Antigen binding site or immunoglobulin or immunoglobulin fragment specific for Bet v 1
QSVLYSSNNKNY

SEQ ID NO: 63 - protein, 3 aa, Artificial Sequence
  Antigen binding site or immunoglobulin or immunoglobulin fragment specific for Bet v 1
WAS

SEQ ID NO: 64 - protein, 9 aa, Artificial Sequence
  Antigen binding site or immunoglobulin or immunoglobulin fragment specific for Bet v 1
QQYYTTPLT

SEQ ID NO: 65 - protein, 132 aa, Artificial Sequence
  Antigen binding site or immunoglobulin or immunoglobulin fragment specific for Bet v 1
MEFGLSWLFLVAFLKGVQCEVQLLESGGGLVQPGGSLRLSCVASGFTFTNYAMTWVRQAPGKGLEWVSAISNYGGSTYYSDSVKGRFTISRDNSKSTLSLHMNSLRAEDTALYYCAKYFDFWGQGTLVTVSS

SEQ ID NO: 66 - protein, 128 aa, Artificial Sequence
  Antigen binding site or immunoglobulin or immunoglobulin fragment specific for Bet v 1
MRLPAQLLGLLLLWVPGAGCDIQLTQSPSSLSASVGDRVTITCRVSQGISNCLNWYRRKPGKVNKVLFYSASNFQSGVPSRFSASASGTDLTLSVNTLQPEDVATCYCQRTDNALHHFAQGTRLEIKR

SEQ ID NO: 67 - protein, 8 aa, Artificial Sequence
  Antigen binding site or immunoglobulin or immunoglobulin fragment specific for Bet v 1
GFTFTNYA

SEQ ID NO: 68 - protein, 8 aa, Artificial Sequence
  Antigen binding site or immunoglobulin or immunoglobulin fragment specific for Bet v 1
ISNYGGST

SEQ ID NO: 69 - protein, 6 aa, Artificial Sequence
  Antigen binding site or immunoglobulin or immunoglobulin fragment specific for Bet v 1
AKYFDF

SEQ ID NO: 70 - protein, 6 aa, Artificial Sequence
  Antigen binding site or immunoglobulin or immunoglobulin fragment specific for Bet v 1
QGISNC

SEQ ID NO: 71 - protein, 9 aa, Artificial Sequence
  Antigen binding site or immunoglobulin or immunoglobulin fragment specific for Bet v 1
QRTDNALHH

SEQ ID NO: 72 - protein, 120 aa, Artificial Sequence
  Antigen binding site or immunoglobulin or immunoglobulin fragment specific for Phl p 5
LESGGGVVQPGRSLRLSCAASEFAFDSYEIYWVRQAPGKGLEWVAMISIDETNKHYADSVKGRFTIFRDNSNKTVHLQMNNLRSEDSGVYYCARWNNWNHRGMVFAFDLWGQGTMVTVSS

SEQ ID NO: 73 - protein, 106 aa, Artificial Sequence; identical to SEQ ID NO: 7
  Antigen binding site or immunoglobulin or immunoglobulin fragment specific for Phl p 5

SEQ ID NO: 74 - protein, 108 aa, Artificial Sequence
  Antigen binding site or immunoglobulin or immunoglobulin fragment specific for Phl p 5
ELVMTQSPSSVSASVGDRVTITCRASHSISNYLNWYQQKPGKAPKLLIYAASSLQSGVPSRFSGSGSGTDFTLTISSLQPEDFANYYCQESFSPSGTFGQGTKVEIKR

SEQ ID NO: 75 - protein, 106 aa, Artificial Sequence
  Antigen binding site or immunoglobulin or immunoglobulin fragment specific for Phl p 5
ELTQSPSSLSASVGDRVTITCRASQSILGYLNWYQQKPGKAPKLLIYAASTLQSGVPSRFSGSGSGTDFTLTITSLQPDDFATYYCQQSYITPRTFGQGTKVEVKR

SEQ ID NO: 76 - protein, 108 aa, Artificial Sequence
  Antigen binding site or immunoglobulin or immunoglobulin fragment specific for Phl p 5
ELVMTQSPSSLSAFVGDRVTITCRASQSISSYLNWYQQKPGKAPKLLIYAASSLQSGVPSRFSGSGSGTDFTLTISSLQPEDFATYYCQQSHSTPYTFGQGTNLEIKR

SEQ ID NO: 77 - protein, 8 aa, Artificial Sequence
  Antigen binding site or immunoglobulin or immunoglobulin fragment specific for Phl p 5
EFAFDSYE

SEQ ID NO: 78 - protein, 8 aa, Artificial Sequence
  Antigen binding site or immunoglobulin or immunoglobulin fragment specific for Phl p 5
ISIDETNK

SEQ ID NO: 79 - protein, 17 aa, Artificial Sequence
  Antigen binding site or immunoglobulin or immunoglobulin fragment specific for Phl p 5
ARWNNWNHRGMVFAFDL

SEQ ID NO: 80 - protein, 6 aa, Artificial Sequence
  Antigen binding site or immunoglobulin or immunoglobulin fragment specific for Phl p 5
QGISSW

SEQ ID NO: 81 - protein, 6 aa, Artificial Sequence
  Antigen binding site or immunoglobulin or immunoglobulin fragment specific for Phl p 5
HSISNY

SEQ ID NO: 82 - protein, 9 aa, Artificial Sequence
  Antigen binding site or immunoglobulin or immunoglobulin fragment specific for Phl p 5
QESFSPSGT

SEQ ID NO: 83 - protein, 6 aa, Artificial Sequence
  Antigen binding site or immunoglobulin or immunoglobulin fragment specific for Phl p 5
QSILGY

SEQ ID NO: 84 - protein, 9 aa, Artificial Sequence
  Antigen binding site or immunoglobulin or immunoglobulin fragment specific for Phl p 5
QQSYITPRT

SEQ ID NO: 85 - protein, 9 aa, Artificial Sequence
  Antigen binding site or immunoglobulin or immunoglobulin fragment specific for Phl p 5
QQSHSTPYT

SEQ ID NO: 86 - protein, 256 aa, Artificial Sequence
  Antigen binding site or immunoglobulin or immunoglobulin fragment specific for Phl p 6
EVQLVQSGGGLVKPGGSLRLSCAASGFIFSDHYMAWVRQTPGKGLEWISYISMTSSYTNYADSVKGRFTISRDNAKNSLSLEMNSLRVEDTAVYYCARDRLRNWNDDAFDIWGPGTMVTVSSGGGGSGGGGSGGGGSSYELTQPASVSGSPGQSITISCTGTPSDIGTYPYVSWYQQHPDRAPKLLIYGVTNRPSGVSNRFSGSKSGNTASLTISELQAEDEADYYCSSYTLTSIVVFGGGTKLTVLGDYKDDDDK

SEQ ID NO: 87 - protein, 122 aa, Artificial Sequence
  Antigen binding site or immunoglobulin or immunoglobulin fragment specific for Phl p 6
EVQLVQSGGGLVKPGGSLRLSCAASGFIFSDHYMAWVRQTPGKGLEWISYISMTSSYTNYADSVKGRFTISRDNAKNSLSLEMNSLRVEDTAVYYCARDRLRNWNDDAFDIWGPGTMVTVSS

SEQ ID NO: 88 - protein, 119 aa, Artificial Sequence
  Antigen binding site or immunoglobulin or immunoglobulin fragment specific for Phl p 6
SYELTQPASVSGSPGQSITISCTGTPSDIGTYPYVSWYQQHPDRAPKLLIYGVTNRPSGVSNRFSGSKSGNTASLTISELQAEDEADYYCSSYTLTSIVVFGGGTKLTVLGDYKDDDDK

SEQ ID NO: 89 - protein, 8 aa, Artificial Sequence
  Antigen binding site or immunoglobulin or immunoglobulin fragment specific for Phl p 6
GFIFSDHY

SEQ ID NO: 90 - protein, 8 aa, Artificial Sequence
  Antigen binding site or immunoglobulin or immunoglobulin fragment specific for Phl p 6
ISMTSSYT

SEQ ID NO: 91 - protein, 15 aa, Artificial Sequence
  Antigen binding site or immunoglobulin or immunoglobulin fragment specific for Phl p 6
ARDRLRNWNDDAFDI

SEQ ID NO: 92 - protein, 9 aa, Artificial Sequence
  Antigen binding site or immunoglobulin or immunoglobulin fragment specific for Phl p 6
PSDIGTYPY

SEQ ID NO: 93 - protein, 3 aa, Artificial Sequence
  Antigen binding site or immunoglobulin or immunoglobulin fragment specific for Phl p 6
GVT

SEQ ID NO: 94 - protein, 10 aa, Artificial Sequence
  Antigen binding site or immunoglobulin or immunoglobulin fragment specific for Phl p 6
SSYTLTSIVV